(19) United States
(12) Patent Application Publication
(10) Pub. No.: US 2020/0370122 A1
(43) Pub. Date: Nov. 26, 2020

(54) IMMUNE INDEX METHODS FOR PREDICTING BREAST CANCER OUTCOME

(71) Applicant: Tong Zhou, Durham, NC (US)

(72) Inventor: Tong Zhou, Durham, NC (US)

(21) Appl. No.: 15/441,686

(22) Filed: Feb. 24, 2017

(51) Int. Cl. C12Q 1/6886

(52) U.S. Cl. CPC C12Q 1/6886; C12Q 2600/158; C12Q 2600/118; C12Q 2600/106

(57) ABSTRACT

Provided are methods for diagnosing and predicting the outcome of a breast cancer patient or related cancers. The methods include determining expression levels of a plurality of biomarkers (selected genes) related to immune function in a biological sample, such as tumor tissues or a body fluid such as blood, from the patient. The expression levels of the biomarkers are used to derive an index which can be used as an indicator predictive of cancer patient outcome. Overexpression of a plurality of biomarkers of the invention can be used to generate a score or value which has been demonstrated herein to be indicative of a good or better patient outcome. The index, generated by the methods of the invention can also be used to stratify cancer subtypes, and also can be combined with conventional clinical parameters to better inform clinical decisions.

### FIELD OF THE INVENTION

This invention relates to measures for predicting a cancer patient's clinical outcome, wherein the measures can be utilized as a value, score, or an index that is generated from the expression levels of a plurality of genes, associated with immunological function, from a patient's biological sample. The index is shown herein to be predictive of the clinical outcome of a patient and thereby aids a physician to make clinical treatment decisions for that patient. The methods for generating the index or score provide indicators that can be used to distinguish cancer patients, including but not limited to breast cancer patients, with a poor prognosis from those with a good prognosis, and further allow the identification of high-risk and low risk, early-stage breast cancer patients with a resultant benefit of being able to choose and apply different anticancer treatments.

### BACKGROUND OF THE INVENTION

Breast cancer is not a single disease, but rather is reflected by its multiple subtypes based on its gene expression profile. A woman in the United States (US) has one in eight chance of developing breast cancer during her lifetime. In 2016, the American Cancer Society estimated 246,660 new cases of invasive breast cancer expected to be diagnosed among US women, as well as an estimated 64,640 additional cases of in situ breast cancer. Approximately 40,450 US women are expected to die from breast cancer annually. Only lung cancer accounts for more cancer deaths in women. Today, approximately 80% of breast cancer cases are diagnosed in the early stages of the disease when survival rates are at their highest. As a result, about 85% percent of breast cancer patients are alive at least five years after diagnosis. Despite these advances, approximately 20% of women diagnosed with early-stage breast cancer have a poor ten-year outcome and will suffer disease recurrence, metastasis, or death within this time period.

In the past decades, methods and factors for assessing breast cancer prognosis and predicting drug response have been used in research and clinical practice. Prognostic parameters include conventional clinical data, such as tumor size, nodal status and histological grade, and molecular markers that provide some information regarding prognosis and likely response to particular treatments. For example, IHC determination of estrogen (ER) and progesterone (PR) steroid hormone receptor status has become a routine procedure in assessment of breast cancer patients. Tumors that are hormone receptor positive are more likely to respond to hormone therapy, and also typically grow less aggressively, thereby resulting in a better prognosis for patients with ER+/PR+ tumors. The methods disclosed herein can be used in combination with assessment of conventional clinical parameters, such as tumor size, tumor grade, lymph node status, and gene expression level of additional biomarkers, such as Her-2 and estrogen and progesterone hormone receptors (see US Patent US20100221722A1). The methods can also stratify or improve the existing diagnosis using commercially available gene profiling systems. However, desired is a more accurate prediction of breast cancer clinical outcome in an attempt to reach “precision diagnosis”.

Metastases are the main cause of mortality for breast cancer patients. Aggressive breast tumors typically metastasize to common sites such as regional axillary lymph nodes, and ultimately to distant organ sites including lung, bone, liver, and brain. Therefore, accurate and sensitive methods for evaluating the metastasis risk of a cancer patient remain an unmet medical need. Current gene profiling methods do not take into consideration the significance of several measures of immune capability in assessing the prognosis or clinical outcome of breast cancer

### SUMMARY OF THE INVENTION

The invention provides methods for evaluating genes related to immunological function to generate scores and an index which can be used as measures or indicators to stratify the prediction of the metastatic potential of a tumor, as well as an indicator of the clinical outcome or prognosis of a patient (e.g., patient survival), all of which are important for decision-making in clinical practice. The methods comprise measuring the expression level of a plurality of immune-related genes comprising of APOBEC3G (Apolipoprotein B mRNA editing enzyme, catalytic polypeptide-like 3G; GenBank Accession No. NM_021822.3; Protein Accession No. NP_068594.1; SEQ ID NO: 1), CCL5 (Chemokine (C-C motif) ligand 5; GenBank Accession No. NM_001278736; Protein Accession No. NP_002976; SEQ ID NO: 2), CCR2 (Chemokine (C-C motif) receptor 2; GenBank Accession No. NM_001194959; Protein Accession No. NP_001116513.2 NP_001116868.1; SEQ ID NO: 3), CD2 (CD2 molecule; GenBank Accession No. NM_001767; Protein Accession No. NP_001758; SEQ ID NO: 4), CD27 (CD27 molecule; GenBank Accession No. NM_001242; Protein Accession No. NP_001233; SEQ ID NO: 5), CD3D (CD3d molecule, delta (CD3-TCR complex); GenBank Accession No. NM_000732; Protein Accession No. NP_001035741; SEQ ID NO: 6), CD52 (CD52 molecule; GenBank Accession No. NM_001803; Protein Accession No. NP_001794; SEQ ID NO: 7), CORO1A (Coronin, actin binding protein, 1A; GenBank Accession No. NM_001193333; Protein Accession No. NP_009005; SEQ ID NO: 8), CXCL9 (Chemokine (C-X-C motif) ligand 9; GenBank Accession No. NM_002416; Protein Accession No. NP_002407; SEQ ID NO: 9), GZMA (Granzyme A (granzyme 1, cytotoxic T-lymphocyte-associated serine esterase 3); GenBank Accession No. NM_006144; Protein Accession No. NP_006135; SEQ ID NO: 10), GZMK (Granzyme K (granzyme 3; tryptase II); GenBank Accession No. NM_002104; Protein Accession No. NP_002095; SEQ ID NO: 11), HLA-DMA (Major histocompatibility complex, class II, DM alpha and beta; GenBank Accession No. NM_006120.3; Protein Accession No. NP_006111.2; SEQ ID NO: 12), IL2RG (Interleukin 2 receptor, gamma; GenBank Accession No. NM_000206; Protein Accession No. NP_000197; SEQ ID NO: 13), LCK (Lymphocyte-specific protein tyrosine kinase; GenBank Accession No. NM_001042771; Protein Accession No. NP_005347; SEQ ID NO: 14), PRKCB (Protein kinase C, beta; GenBank Accession No. NM_002738; Protein Accession No. NP_997700; SEQ ID NO: 15), PTPRC (Protein tyrosine phosphatase, receptor type, C; GenBank Accession No. NM_001267798; Protein Accession No. NP_563578; SEQ ID NO: 16), and SH2D1A (SH2 domain containing 1A; GenBank Accession No. NM_001114937; Protein Accession No. NP_002342; SEQ ID NO: 17).

Also provided is an “Immune index” for evaluating the prognosis of a breast cancer patient, or a patient having a solid non lymphoid tumor of a tissue/organ type other than breast cancer, are described herein. The Immune index is derived from the values for the levels of gene expression of at least six immune-related genes (which are also referred to as biomarkers) from a panel of biomarkers which comprise seventeen biomarkers selected from the group consisiting of APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A in a biological sample obtained from the cancer patient. Overexpression of a plurality of six or more of the biomarkers of the invention is predictive of a good or better prognosis, meaning a lower risk of cancer recurrence, metastasis or death of a cancer patient caused by the patient's cancer, as compared to the clinical outcome or prognosis of a cancer patient in which the plurality of six or more biomarkers are not over expressed.

In one embodiment, provided is a panel of biomarkers, the expression levels of which can be used to generate scores, measures or an index that can be used to predict the clinical outcome of a breast cancer, response to treatment, and metastatic potential. The panel may comprise a number of the biomarkers of the invention ranging from six biomarkers up to seventeen biomarkers (e.g., 6, 7, 8, 9, 10, 11, 12, 13, 14, 15, 16 or 17 biomarkers).

Provided are methods which allow distinguishing breast cancer patients with a better prognosis from breast cancer patients with a worse prognosis. The methods of the invention comprise determining the levels of gene or protein expression of a panel of biomarkers of the invention from a biological sample from an individual suspected of having or diagnosed as having cancer, and converting the expression levels to a score or index which can then be used as an indicator in predicting clinical outcome, or prognosis of the individual. Expression levels of biomarkers can be determined using a variety of technologies known in the art that include, but are not limited to, gene expression microarrays, Next Generation Sequencing (NGS), Targeted RNA expression sequencing, polymerase chain reaction (PCR), antibody-based detection, and proteomics. However, in most cases biomarker expression is usually assessed at the protein level or nucleic acid level.

### DETAILED DESCRIPTION OF THE INVENTION

Overview

The present invention describes methods for evaluating breast cancer prognosis at relatively early stage or for analyzing retrospective data for medical usage or clinical practice. The first step is to measure gene expression levels of a plurality of biomarkers of the invention, in a biological sample obtained from a cancer patient. The biological sample may comprise a body fluid (e.g., blood) or fraction thereof (e.g., serum or plasma) or tumor tissue. The tumor tissue sample can be tumor tissue obtained via surgery, biopsy, or any other method. The biomarkers of the invention comprise a panel of a plurality of immune-related genes selected from the group of genes consisting of APOBEC3G, CCL5, CCR2, CD2, CD27, CD3D, CD52, CORO1A, CXCL9, GZMA, GZMK, HLA-DMA, IL2RG, LCK, PRKCB, PTPRC, and SH2D1A (as represented by SEQ ID NOs: 1-17, respectively), wherein the expression levels of at least six of the genes is converted into a score or index that can be used as a predictor for clinical outcomes or prognosis.

In one embodiment, the method comprises determining the expression levels of the RNA transcripts or their expression products of at least six biomarkers selected from a panel or an “immune index” group of genes consisting of APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A in a biological sample obtained from a cancer patient. Biomarker expression can be normalized against the expression levels of all RNA transcripts or their expression products in the biological sample, or against a group of housekeeper gene's RNA transcripts or their expression products in the sample. The gene expression level of a portion (six or more) or all of the seventeen biomarkers are related to a patient's prognosis or risk of distant metastasis. As known to those skilled in the art, RNA transcripts present in a sample can be transformed into cDNA which is amplified and detected relative to the amount of the respective RNA transcript present in the sample.

In another embodiment, the method comprises detecting expression of at least six biomarkers selected from the immune index group of genes consisting of APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A in a biological sample from the patient. Detection in the biological sample (e.g., cancer tissue or circulating blood) and determination of overexpression of at least six of such biomarkers, relative to expression in a healthy individual or a cancer patient having a poor prognosis, are used to generate scores or an index which have been shown herein to correlate with a comparatively good or better prognosis for the patient (than a cancer patient in which the biomarkers are not overexpressed).

The methods, scores and index of the invention can also be used to assist in selecting appropriate therapy and to identify patients that would benefit from more or less aggressive therapy based on the immune index value determined by a panel of at least six or up to all of seventeen biomarkers comprising APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A (as represented by SEQ ID NOs: 1-17). Overexpression of at least six biomarkers of the panel allows breast cancer patients with good or better prognosis to be distinguished from those who may have higher probability to develop distant metastasis or who carry a poor or worse prognosis.

The term “breast cancer” means any malignancy of the breast tissue including but not limited to, carcinomas and sarcomas. Breast cancer may include Ductal carcinoma in situ (DCIS), lobular carcinoma in situ (LCIS), mucinous carcinoma, infiltrating ductal (IDC), and infiltrating lobular carcinoma (ILC). In most embodiments of the invention, the individual of interest is a patient diagnosed with breast cancer or a patient (e.g., determined by genetic factors and/or familial incidence to be at high risk) to be screened for breast cancer.

The current invention aims to provide more accurate test termed as precision diagnosis at molecular genetic level but not to replace the routine methods in clinical practice. The standardized breast cancer staging TNM system was developed by The American Joint Committee on Cancer (AJCC). Patients are assessed for primary tumor size (T), regional lymph node status (N), and the presence/absence of distant metastasis (M) and then classified into stages 0-IV based on this combination of factors. In this system, primary tumor size is categorized on a scale of 0-4 (T0: no evidence of primary tumor; T1:<=2 cm; T2:>2 cm-<=5 cm; T3:>5 cm; T4: tumor of any size with direct spread to chest wall or skin). Lymph node status is classified as N0-N3 (NO: regional lymph nodes are free of metastasis; N1: metastasis to movable, same-side axillary lymph node(s); N2: metastasis to same-side lymph node(s) fixed to one another or to other structures; N3: metastasis to same-side lymph nodes beneath the breastbone). Metastasis is categorized by the absence (M0) or presence of distant metastases (M1). Routine methods of identifying breast cancer patients and staging the disease may comprise or combine manual examination, biopsy, review of a patient's family history, and imaging technologies including mammography, magnetic resonance imaging (MRI), and positron emission tomography (PET).

The term “Prognosis” means a patient's “outcome predictions” or “outcome” and the likely course or probability of disease recurrence or disease progression including, for instance, probability of disease remission, disease relapse, tumor recurrence, metastasis, and even patient's death resulting in from the underlying tumor. The term “good prognosis” or “better prognosis” means “good outcome” or “better outcome” and the likelihood that a cancer patient will remain disease-free or cancer-free for a period of time. On the opposite, “poor prognosis” or “worse prognosis” means “poor outcome” or “worse outcome” and a higher likelihood of a relapse or recurrence of the patient's underlying cancer or tumor, metastasis, or even cancer-related death. In some embodiments, for instance the time frame for assessing prognosis and outcome is less than one year to twenty years, or even more years in rare cases. While there are a number of time parameters known to those skilled in the art, typically months or years are used to refer to remission, mortality rate (e.g., 5 year mortality rate), and survival rate. As routine, the relevant time for assessing prognosis or disease free survival time begins with the surgical removal of the tumor or the start of therapy for suppression, mitigation, or inhibition of tumor growth (“anticancer treatment”). Thus, for example, in particular embodiments, a good prognosis or better prognosis refers to the likelihood that a breast cancer patient will remain free of the underlying cancer or tumor for a period of time specified usually in number of years. Such patients may be eligible for fewer cycles of anticancer treatment (“less aggressive treatment”) as compared to patients having a poor prognosis. In further aspects of the invention, a poor prognosis or worse prognosis refers to the likelihood that a breast cancer patient will experience disease relapse, tumor recurrence, metastasis, or death within less than the specified years. Time frames for assessing prognosis and outcome can be various in each individual case or studying cohort.

In some embodiments described herein, prognostic performance of the biomarkers and/or other clinical parameters was assessed utilizing a Kaplan-Meier Survival Analysis. Methods for assessing statistical significance are usually using Kaplan Meier curves. In statistic analysis, a p-value of equal to or less than 0.05 is deemed to be statistical significant. In the current invention, p-values are used as indicators for most survival analysis.

Clinical and prognostic parameters for breast cancer are routinely used to predict treatment outcome and the likelihood of disease recurrence or even distant metastasis. Those parameters are lymph node status, tumor size, histologic grade, estrogen (ER) and progesterone (PR) hormone receptor status, Her-2 levels (IHC). An “estrogen receptor-positive patient” displays ER expression in a breast tumor, whereas an “estrogen receptor-negative patient” does not. Using the methods of the present invention, the prognosis of a breast cancer patient can be determined independent of or in combination with assessment of one or more of these or other clinical and prognostic parameters. In some embodiments, combining the methods described herein with evaluation of other clinical or prognostic parameters allows a more precise determination of breast cancer prognosis to achieve precise medicine individually. For example, the methods of the invention may be combined with analysis of routine methods such as ER, PR, and Her-2 expression levels or other methods in clinical practice. In some embodiments, patient data obtained via the methods described herein may be incorporated with analysis of clinical information and existing commercially available tests for assessing breast cancer prognosis. Patients assessed with poor prognosis may be qualified for more aggressive breast cancer treatment.

Breast cancer is managed by several alternative strategies for anticancer treatment that may comprise or combine some of the methods such as surgery, radiation therapy, hormone therapy, chemotherapy. As known, treatment decisions for individual breast cancer patients can be based on endocrine responsiveness of the tumor, menopausal status of the patient, the location and number of patient lymph nodes involved, estrogen and progesterone receptor status of the tumor, primary tumor size, patient age, and stage of the disease at diagnosis. Analysis of a variety of clinical factors and clinical trials has led to the development of recommendations and treatment guidelines for early-stage breast cancer by the International Consensus Panel of the St. Gallen Conference (Vienna, Austria 18-21 Mar. 2015) (Coates A S eta al. Tailoring therapies—improving the management of early breast cancer: St Gallen International Expert Consensus on the Primary Therapy of Early Breast Cancer 2015. Anna Oncology 2015). The 14th St. Gallen International Breast Cancer Conference (2015) reviewed substantial new evidence on locoregional and systemic therapies for early breast cancer. Further experience has supported the adequacy of tumor margins defined as ‘no ink on invasive tumor or DCIS’ and the safety of omitting axillary dissection in specific cohorts. Radiotherapy trials support irradiation of regional nodes in node-positive disease. For the treatment of HER2-positive disease in patients with node-negative, cancers up to 1 cm, the Panel endorsed a simplified regimen comprising paclitaxel and trastuzumab (Herceptin®, Genentech, South San Francisco, Calif.) without anthracycline as adjuvant therapy. For premenopausal patients with endocrine responsive disease, the Panel endorsed the role of ovarian function suppression with either tamoxifen or exemestane for patients at higher risk. The Panel noted the value of an LHRH agonist given during chemotherapy for premenopausal women with ER negative disease in protecting against premature ovarian failure and preserving fertility. The Panel noted increasing evidence for the prognostic value of commonly used multi-parameter molecular markers, some of which also carried prognostic information for late relapse. The Panel noted that the results of such tests, where available, were frequently used to assist decisions about the inclusion of cytotoxic chemotherapy in the treatment of patients with luminal disease, but noted that threshold values had not been established for this purpose for any of these tests. Multiple parameter molecular assays are expensive and therefore unavailable in much of the world. The majority of new breast cancer cases and breast cancer deaths now occur in less developed regions of the world. In these areas, less expensive pathology tests may provide valuable information. The Panel recommendations on treatment are not intended to apply to all patients, but rather to establish norms appropriate for the majority. In particular embodiments, the methods of the present invention may be used in conjunction with the treatment guidelines established by the St. Gallen Conference to permit physicians to make more informed breast cancer treatment decisions.

The methods of the invention have particular use in choosing appropriate treatment for early-stage breast cancer, as well as for predicting the likelihood of survival of a breast cancer patient. In particular, the methods may be used predict the likelihood of long-term, disease-free survival. By “predicting the likelihood of survival of a breast cancer patient” is intended assessing the risk that a patient will die as a result of the underlying breast cancer. “Long-term, disease-free survival” is intended to mean that the patient does not die from or suffer a recurrence of the underlying breast cancer within a period of at least five years, or for a longer period such as at least ten or more years, following initial diagnosis or treatment. Such methods for predicting the likelihood of survival of a breast cancer patient include detecting expression of at least six genes selected from the group of genes consisting of APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A, in a biological sample from the patient, where overexpression of a plurality of these biomarkers is indicative better survival. Probability of survival can be assessed in comparison to, for example, breast cancer survival statistics available in the combined data set. The method further comprises converting the levels of overexpression to a score or index which can then be used as the indicator for predicting clinical outcome or prognosis.

The present methods for evaluating breast cancer prognosis can also be combined with other prognostic methods such as assessment of conventional clinical factors, such as tumor size, tumor grade and lymph node status as well as additional molecular markers known in the art such as estrogen and progesterone hormone receptors, Her-2 and p53 and microarrays such as Agilent (van′t Veer et al., N. Engl. J. Med. 347:1999-2009, 2002) and Affymetrix (Pawitan et al., Cancer Res. 7: 953-64, 2005) and most advanced RNA-seq such as TCGA RNA-seq (TCGA. Nature 490: 61-70, 2012) for purposes of selecting an appropriate breast cancer treatment.

In certain embodiments, methods scores, and index provide an additional or alternative treatment decision-making factor. The methods scores, and index of the invention permit the differentiation of breast cancer patients with a good prognosis from those cancer patients more likely to suffer a recurrence (e.g., having a “poor prognosis”).

The biomarkers of the invention include genes and proteins. Such biomarkers include DNA comprising the entire or partial sequence of the nucleic acid sequence encoding the biomarker, or the complement of such a sequence. The biomarker nucleic acids also include RNA comprising the entire or partial sequence of any of the nucleic acid sequences of interest. A biomarker protein is a protein encoded by or corresponding to a DNA biomarker of the invention. A biomarker protein comprises the entire or partial amino acid sequence of any of the biomarker proteins or polypeptides. Fragments and variants of biomarker genes and proteins are refer to an increased likelihood of relapse or recurrence of the underlying cancer or tumor, metastasis, or death within ten years, such as five years. In other aspects of the invention, the absence of overexpression of a biomarker or combination of biomarkers of interest is indicative of a worse prognosis. As used herein, “indicative of a good prognosis” refers to an increased likelihood that the patient will remain cancer-free.

A “biomarker” is a gene or protein with a level of expression in a tissue or cell is altered compared to that of a normal or healthy cell or tissue. The biomarkers of the present invention are genes and proteins whose overexpression correlates with cancer, particularly breast cancer, prognosis. As used herein, “overexpression” means expression greater than the expression detected in normal, non-cancerous tissue. For example, an RNA transcript or its expression product that is overexpressed in a cancer cell or tissue may be expressed at a level that is 1.5 times higher than in a normal, non-cancerous cell or tissue, such as 2 times higher, 3 times higher, 5 times higher, or more than 5 times higher.

In some embodiments, overexpression, such as of an RNA transcript or its expression product, is determined by normalization to the level of reference RNA transcripts or their expression products, which can be all measured transcripts (or their products) in the sample or a particular reference set of RNA transcripts (or their products). Normalization is performed to correct for or normalize away both differences in the amount of RNA assayed and variability in the quality of the RNA used. Therefore, a method of the invention comprises assaying for expression levels of a panel of immune-related genes of the invention. Although the methods of the invention require the detection and quantification of expression of at least six biomarkers in a patient sample for evaluating breast cancer prognosis, 7, 8, 9, 10, 11, 12, 13, or more biomarkers may be used to practice the present invention, including to derive an immune index or score of predictive value.

In particular embodiments, selective overexpression of a panel of biomarkers or combination of biomarkers of interest in a patient sample is indicative of a good or better cancer prognosis. By “indicative of a good or better prognosis” is intended that overexpression of the particular biomarker or combination of biomarkers is associated with a lower probability of relapse or recurrence of the underlying cancer or tumor, metastasis or patient's death.

Biomarkers

The biomarkers of the present invention are selected and intended a portion of the polynucleotide or a portion of the amino acid sequence and hence protein encoded thereby. Polynucleotides that are fragments of a biomarker nucleotide sequence generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a full-length biomarker polynucleotide disclosed herein. A fragment of a biomarker polynucleotide will generally encode at least 15, 25, 30, 50, 100, 150, 200, or 250 contiguous amino acids, or up to the total number of amino acids present in a full-length biomarker protein of the invention. “Variant” is intended to mean substantially similar sequences. Generally, variants of a particular biomarker of the invention Will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that biomarker as determined by sequence alignment programs. A representative oligo polynucleotide sequence for each of the seventeen immune-related genes is provided in the “SEQUENCE LISTING”; however, those skilled in the art can appreciate that known variants of each of these gene sequences may also be utilized in the methods of the invention.

Sample Source

In particular embodiments, the methods for evaluating breast cancer prognosis include collecting a patient biological sample having a cancer cell or tissue, such as a breast tissue sample or a primary breast tumor tissue sample. By “biological sample” is intended any sampling of cells, tissues, or bodily fluids such as blood in which expression of a biomarker can be detected. Examples of such biological samples involving tissue or cells include, but are not limited to, biopsies and smears. Bodily fluids useful in the present invention include blood, lymph, urine, saliva, nipple aspirates, gynecological fluids, or any other bodily secretion or derivative thereof. Blood can include whole blood, plasma, serum, or any derivative of blood. In some embodiments, the biological sample includes breast cells, particularly breast tissue from a biopsy, such as a breast tumor tissue sample. Biological samples may be obtained from a patient by a variety of techniques including, for example, by scraping or swabbing an area, by using a needle to aspirate cells or bodily fluids, or by removing a tissue sample such as biopsy. Methods for collecting various biological samples are well known in the art. In some embodiments, a breast tissue sample is obtained by, for example, fine needle aspiration biopsy, core needle biopsy, or excisional biopsy. Fixative and staining solutions may be applied to the cells or tissues for preserving the specimen and for facilitating examination. Biological samples including blood samples, particularly breast tissue samples, may be transferred to a glass slide for viewing under magnification. In one embodiment, the biological sample is a formalin-fixed, paraffin-embedded breast tissue sample, particularly a primary breast tumor sample.

Any method available in the art for detecting expression of biomarkers are elaborated further herein. The expression of a biomarker of the invention can be detected on a nucleic acid level such as an RNA transcript or a protein level. By “detecting expression” is intended determining the quantity or presence of an RNA transcript (representing a gene or its variant) or its expression product of a biomarker gene. Thus, “detecting expression” encompasses instances where a biomarker is determined not to be expressed, not to be detectably expressed, under expressed, expressed at a normal level, or overexpressed. In order to determine overexpression, the biological sample to be examined can be compared with a corresponding biological sample that originates from a healthy person. That is, the “normal” level of expression is the level of expression of the biomarker in, for example, a breast tissue sample from a human subject or patient not afflicted with breast cancer. Reference values for such expression are known to those skilled in the art. Such a sample can be present in standardized form. In some embodiments, determination of biomarker overexpression requires no comparison between the biological sample and a corresponding biological sample that originates from a healthy person. For example, detection of overexpression of a plurality of biomarkers of the invention is indicative of a good or better prognosis in a breast tumor sample may preclude the need for comparison to a corresponding breast tissue sample that originates from a healthy person. Moreover, in some aspects of the invention, no expression, under expression, or normal expression of a biomarker or combination of biomarkers of interest provides useful information regarding the prognosis of a breast cancer patient.

Methods for detecting expression of the biomarkers of the invention, that is, gene expression profiling, include methods based on hybridization analysis of polynucleotides, methods based on sequencing of polynucleotides such as NGS, immunohistochemistry (IHC) methods, and proteomics-based methods. The most commonly used methods known in the art for the quantification of mRNA expression in a sample include northern blotting and in situ hybridization, RNAse protection assays, PCR-based methods, such as reverse transcription PCR (RT-PCR), and array-based methods. Alternatively, antibodies may be employed that can recognize specific duplexes, including DNA duplexes, RNA duplexes, and DNA-RNA hybrid duplexes, or DNA-protein duplexes. Representative methods for sequencing-based gene expression analysis include Serial Analysis of Gene Expression (SAGE) and gene expression analysis by massively parallel signature sequencing. Thus, determination of expression levels of a biomarker may be via the detection of levels of, a nucleotide transcript or a protein encoded by or corresponding to the biomarker. Probes can be synthesized by one of skill in the art, or derived from appropriate biological preparations. Probes may be specifically designed to be labeled. Examples of molecules that can be utilized as probes include, but are not limited to, RNA, DNA, proteins, and antibodies. Illustrative examples of probes that may be used in determining the expression levels of the immune-related genes of the invention may include, but are not limited to oligonucleotides comprising a nucleic acid sequence selected from the group consisting of SEQ ID NOs: 18-34.

Hybridization Analysis of Polynucleotides

In some embodiments, the expression of a biomarker of interest is detected at the nucleic acid level. Nucleic acid-based techniques for assessing expression are well known in the art and include, for example, determining the level of biomarker RNA transcripts (i.e., mRNA) in a biological sample. Many expression detection methods use isolated RNA. The starting material is typically total RNA isolated from a biological sample, such as a tumor or tumor cell line, and corresponding normal tissue or cell line, respectively. Thus RNA can be isolated from a variety of primary tumors, including breast, lung, colon, prostate, brain, liver, kidney, pancreas, spleen, thymus, testis, ovary, uterus, and the like, or tumor cell lines. If the source of mRNA is a primary tumor, mRNA can be extracted, for example, from frozen or archived paraffin-embedded and formalin-fixed tissue samples.

General methods for mRNA extraction are well known in the art and are disclosed in standard textbooks of molecular biology, such as methods described in Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biolechniques 18:42-44, 1995). RNA isolation can also be performed using a purification kit, a buffer set and protease from commercial manufacturers, such as commercially available RNA purification kits, magnetic bead based RNA and DNA isolation kits, (Epicentre, Madison, Wis.), and a Paraffin Block RNA Isolation Kit. RNA prepared from a tumor can be isolated, for example, by cesium chloride density gradient centrifugation, or other standard techniques known in the art.

Isolated mRNA can be used in hybridization or amplification assays that include, not limited to, Southern or Northern analyses, PCR analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250, or 500 nucleotides in length and sufficient to specifically hybridize under stringent conditions to an mRNA or genomic DNA encoding a biomarker of the present invention.

The term “probe” refers to any molecule that can hybridize with the nucleotide sequence (RNA or DNA) corresponding to the biomarker inselectively binding to a specific sequence of the biomarker.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probes are immobilized on a solid surface and the mRNA is contacted with the probes, for example, in a gene chip array. A skilled artisan can readily adapt known mRNA detection methods for use in detecting the level of mRNA encoded by the biomarkers of the present invention.

An alternative method for determining the level of biomarker mRNA in a sample involves the process of nucleic acid amplification, for example, by RT-PCR (U.S. Pat. No. 4,683,202), ligase chain reaction (Barany, Proc. Natl. Acad. Sci. USA 88:189-93, 1991), automatic sequence replication (Guatelli et al., Proc. Natl. Acad. Sci. USA 87:1874-78, 1990), transcriptional amplification system (Kwoh et al., Proc. Natl. Acad. Sci. USA 86:1173-77, 1989), Q-Beta Rep example, U.S. Pat. Nos. 5,856,174 and 5,922,591.
Illustrative methods for determining expression levels include dual color fluorescence, separately labeled circle replication, or any other nucleic acid amplification method, followed by the detection of the amplified molecules using techniques well known to those of skill in the art. These detection schemes are especially useful for the detection of nucleic acid molecules if such molecules are present in very low concentrations. In particular aspects of the invention, biomarker expression is assessed by quantitative fluorogenic RT-PCR. For PCR analysis, methods are available in the art for the determination of primer sequences for use in the analysis. Standard software can then be used for quantification from the detected signal.

Biomarker expression levels of RNA may be monitored using a membrane blot (such as used in hybridization analysis such as Northern, Southern, dot, and the like), or micro-wells, sample tubes, gels, beads, or fibers (or any solid support comprising bound nucleic acids). See, for example, and U.S. Pat. No. 5,445,934. The detection of biomarker expression may also comprise using nucleic acid probes in solution. cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels. Microarray analysis can be performed by commercially available equipment, following manufacturer's protocols. The development of microarray methods for large-scale analysis of gene expression makes it possible to search systematically for molecular markers of cancer classification and outcome prediction in a variety of tumor types.

NGS is particularly useful in detecting expression level of biomarkers. For instance, Targeted RNA-seq are used to detect biomarker expression. Targeted RNA-seq are particularly well suited for this purpose because of the reproducibility between different experiments. Targeted RNA-seq can measure simultaneously the expression levels of large numbers of genes as well as large number of samples called “multiplexing”. Targeted RNA-seq are particularly useful for determining the gene expression profile for a large number of RNAs in multiple samples.

Immunohistochemistry

Immunohistochemistry methods are also suitable for detecting the expression levels of the biomarkers of the present invention. In one embodiment, a patient breast tissue sample is collected by, for example, biopsy techniques known in the art. Samples can be frozen for later preparation or immediately placed in a fixative solution. Tissue samples can be fixed by treatment with a reagent, such as formalin, gluteraldehyde, methanol, or the like and embedded in paraffin. Methods for preparing slides for immunohistochemical analysis from formalin-fixed, paraffin-embedded tissue samples are well known in the art.

In some instances, samples may need to be modified in order to make the biomarker antigens accessible to antibody binding. For example, formalin fixation of tissue samples results in extensive cross-linking of proteins that can lead to the masking or destruction of antigen sites and, subsequently, poor antibody staining As used herein, “antigen retrieval” or “antigen unmasking” refers to methods for increasing antigen accessibility or recovering antigenicity in, for example, formalin-fixed, paraffin-embedded tissue samples. Any method for making antigens more accessible for antibody binding may be used in the practice of the invention, including those antigen retrieval methods known in the art. In particular embodiments, at least five antibodies directed to five distinct biomarkers are used to evaluate the prognosis of a breast cancer patient. Where more than one antibody is used, these antibodies may be added to a single sample sequentially as individual antibody reagents, or simultaneously as an antibody cocktail. Alternatively, each individual antibody may be added to a separate tissue section from a single patient sample, and the resulting data pooled. For detection of protein levels, one can use commercially available antibodies specific for the gene products (proteins) produced by the immune-related genes of the invention.

Antigen retrieval methods include but are not limited to treatment with proteolytic enzymes (e.g., trypsin, chymotrypsin, pepsin, pronase, and the like) or antigen retrieval solutions. Antigen retrieval solutions of interest include, for example, citrate buffer, pH 6.0, Tris buffer, pH 9.5, EDTA, pH 8.0, L.A.B. (“Liberate Antibody Binding Solution, citrate buffer solution, pH 4.0, a detergentsolution, deionized Water, and 2% glacial acetic acid. In some embodiments, antigen retrieval comprises applying the antigen retrieval solution to a formalin-fixed tissue sample and then heating the sample in an oven (e.g., at 60° C.), steamer (e.g., at 95° C.), or pressure cooker (e.g., at 1200 C.) at specified temperatures for defined time periods. In other aspects of the invention, antigen retrieval may be performed at room temperature. Incubation times will vary with the particular anti gene retrieval solution selected and with the incubation temperature. For example, an antigen retrieval solution may be applied to a sample for as little as 5, 10, 20, or 30 minutes or up to overnight. The design of assays to determine the appropriate antigen retrieval solution and optimal incubation times and temperatures is standard and well within the routine capabilities of those of ordinary skill in the art.

Following antigen retrieval, samples are blocked using an appropriate blocking agent (e.g., hydrogen peroxide). An antibody directed to a biomarker of interest is then antibodies and for selecting appropriate antibodies are known in the art. In some embodiments, commercial antibodies directed to specific biomarker proteins can be used to practice the invention. The antibodies of the invention can be selected on the basis of desirable staining of histological samples. That is, the antibodies are selected with the end sample type (e. g., formalin-fixed, paraffin-embedded breast tumor tissue samples) in mind and for binding specificity.

Techniques for detecting antibody binding are well known in the art. Antibody binding to a biomarker of interest can be detected through the use of chemical reagents that generate a detectable signal that corresponds to the level of antibody binding, and, accordingly, to the level of biomarker protein expression. For example, antibody binding can be detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-anti body binding site, thereby resulting in cell or tissuestaining that corresponds to expression level of the biomarker of inter est. Enzymes of particular interest include horseradish per oxidase (HRP) and alkaline phosphatase (AP). Commercial antibody detection systemscan be used to practice the present invention.

The terms “antibody” and “antibodies” broadly encompass naturally occurring forms of antibodies and recombinant antibodies such as single-chain antibodies, chimeric and humanized antibodies and multi-specific antibodies as well as fragments and derivatives of all of the foregoing, which fragments and derivatives have at least an antigenic binding site. Antibody derivatives may comprise a protein or chemical moiety conjugated to the antibody. The antibodies used to practice the invention are selected to have specificity for the biomarker proteins of interest.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, [3-galactosidase, and acetylcholinesterase. Examples of suitable prosthetic group complexes include streptavidin/biotin and avidin/biotin. Examples of suitable fluorescent materials include umbelliferone, fluorescein, fluoresceinisothiocyanate, rhodamine, dichlorotriaziny lamine fluorescein, dansyl chloride, and phycoerythrin. An example of a luminescent material is luminol. Examples of bioluminescent materials include luciferase, luciferin and aequorin. Examples of suitable radioactive materials include 1251′ 1311′ 35S′ and 3H′

In regard to detection of antibody staining in the immunohistochemistry methods of the invention, there also exist in the art, video-microscopy and software methods for the quantitative determination of an amount of multiple molecular species (e.g., biomarker proteins) in a biological sample where each molecular species present is indicated by a representative dye marker having a specific color. Such methods are also known as a colorimetric analysis methods. In these methods, video-microscopy is used to provide an image of the biological sample after it has been stained to visually indicate the presence of a particular biomarker of interest, such as U.S. Pat. Nos. 7,065,236 and 7,133,547, which disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is deconstructed into its component color parts.

### Example 1

Methods

Microarray Data and Data Analysis

Affimetrix® probe level intensity CEL files and their clinical information for two thousands and thirty four patients were downloaded from public database including fourteen cohorts: 1. GSE11121 (Schmidt M et al. Cancer Res 2008; 68[13]:5405-13. PMID: 18593943); 2.GSE12093

(Zhang Y. Breast Cancer Res Treat 2009; 116[2]:303-9. PMID: 18821012); 3. GSE1456 (Pawitan Y et al. Breast Cancer Res 2005; 7[6]:R953-64. PMID: 16280042); 4.GSE2034 (Wang Y et al. Gene-expression profiles to predict distant metastasis of lymph-node-negative primary breast cancer. Lancet2005; 365[9460]:671-9. PMID: 15721472); 5. GSE2603 (Minn A J et al. Nature 2005; 436[7050]:518-24. PMID: 16049480); 6. GSE3494 (Miller L D et al. Proc Natl Acad Sci USA 2005; 102[38]:13550-5. PMID: 16141321); 7.GSE4922 (Ivshina A V et al. Cancer Res 2006; 66[21]:10292-301. PMID: 17079448); 8. GSE5327 (Minn A J et al. Proc Natl Acad Sci USA 2007; 104[16]:6740-5. PMID: 17420468); 9. GSE6532 (Loi S et al. Proc Natl Acad Sci USA 2010; 107[22]:10208-13. PMID: 20479250) and Loi S et al. J Clin Oncol 2007; 25[10]:1239-46. PMID: 17401012); 10. GSE7378 (Yau C et al. Breast Cancer Res 2008; 10[4]:R61. PMID: 18631401); 11.GSE7390 (Desmedt C et al. Clin Cancer Res 2007; 13[11]:3207-14. PMID: 17545524); 12. GSE8193 (Yau C et al. Breast Cancer Res 2007; 9[5]:R59. PMID: 17850661); 13. GSE9195 (Loi S et al. BMC Genomics 2008; 9:239. PMID: 18498629 and Loi S et al. Proc Natl Acad Sci USA 2010; 107[22]:10208-13. PMID: 20479250); 14. ArrayExpress I E-TABM-158(Genomic and transcriptional aberrations linked to breast cancer pathophysiologies. Chin K et al. 2006; Dicer, Drosha, and Outcomes in Patients with Ovarian Cancer. Merritt William M at al. and Gene expression profile analysis of t1 and t2 breast cancer reveals different activation pathways. Riis M L et al. Europe PMC 23533813). The downloaded individual CEL files were first processed by Robust Multi-chip Average (RMA)(Irizarry R A et al. Biostatistics4[2]:249-64, 2003) and then merged into data of 2034 patients which were further batch corrected using Combat (Johnson W E et al. Biostatistics 8(1):118-127, 2007) with subtype as covariate.Ten-fold CV includes different statistical predictors including PAM (Tibshirani et al., Proc. Natl. Acad. Sci. USA 99:6567-72, 2002), a k-Nearest Neighbor Classifier (KNN) with either Euclidean distance or one-minus-Spearman-correlation as the distance function and a Class Nearest Centroid (CNC) metric with either Euclidean distance or one-minus Spearman-correlation as the distance function. Univariate Kaplan-Meier survival analysis was performed using WINSTAT for EXCEL® (R. Fitch Software, Lehigh Valley, Pa.). EPIG program was used to select genes related to cancer immunology (Chou J W et al. BMC Bioinformatics 8:427, 2007 and Zhou T et al. Environmental Health Perspectives 114: 553-559). Algorithm for “intrinsic subtype” assignment was described in Fan et al. (N. Engl. J. Med. 355:560-69, 2006). However, we included “Immuno” subtype as a novel subtype.

An immune index of the invention is an average expression value across a plurality of the immune-related genes (six or more of: APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A). Briefly, the Affymetrix gene expression data in a format of CEL files were first processed using Robust Multi-chip Average (RMA)(Irizarry R A et al. Biostatistics4[2]:249-64, 2003) and then merged into a dataset which were further batch corrected using Combat (Johnson W E et al. Biostatistics 2007; 8(1):118-127) and then column standardized within a single study (Bolstad et al. Bioinformatics 19:185-193, 2003) or cross platform (Shabalin A A et al. Bioinformatics 24 (9): 1154-1160, 2008). Next, the expression values of the selected immune-related genes were extracted, and the average of those genes' expression value for each sample was an “immune index”. For immune index group division, the patients were divided into a two group (iweak and istrong) classification based their immune index and using the cut off values that were identified using X-tile (Camp et al., Clin. Cancer Res. 10:7252-59, 2004). For instance, the breast tumor sample GEO|GSE11121|GSM282380 had final gene expression values of the seventeen genes APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A were 1.14, 1.442, 0.798, 1.293, 1.037, 1.464, 1.494, 1.257, 2.538, 1.758, 1.69, 0.744, 1.482, 1.058, 1.073, 1.082, 1.07 separately, and the immune index was the average (1.32) of the seventeen genes. This average value 1.32 was larger than 0; hence, the sample GEO|GSE11121|GSM282380 was categorized into “istrong” group. This patient's was distance metastasis free at 7 years follow up and was node negative (see Table 3). The immune index was also validated in a number of independent test sets, such as 337 patients assayed on Agilent microarrays (NK1337. Chang et al., Proc. Natl. Acad. Sci. USA 102:3738-43, 2005), another test set of patients assayed on Affymetrix microarrays, and TCGA gene expression dataset (TCGA. Nature 490: 61-70, 2012) using RNA-seq (NGS). To perform these across data set analyses, for the NKI337 dataset the log ratio of red channel intensity versus green channel intensity was used and the data was median centered for every gene across the 337 arrays. The NKI337 dataset was normalized by Distance weighted Discrimination (DWD) (Benito et al., Bioinformalics 20: 105-14, 2004) and then column standardized. For the Affymetrix dataset the probe level intensity CEL files were processed by routine Robust Multi-chip Average (RMA). The probe sets log intensity was median centered for every gene across all the arrays. The Affymetrix dataset was then normalized and column standardized.

Results

Identification of Immuno Subtypes

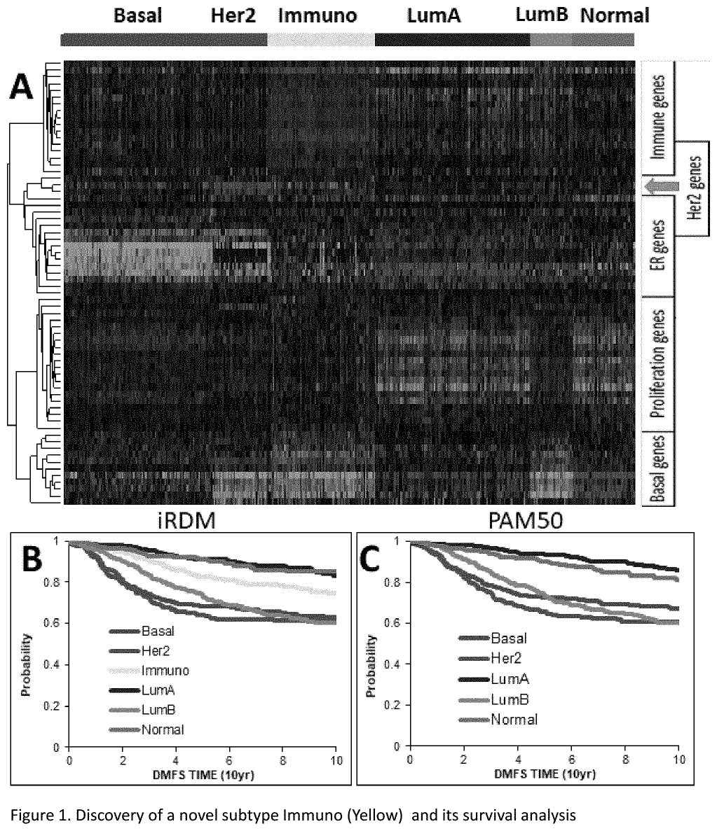

Immune index stratified breast cancer intrinsic subtypes. In addition to the classic five breast cancer subtypes (Perou et al., Nature 406:747 52, 2000; Sorlie et al., Proc. Natl. Acad. Sci. USA 100:8418-23, 2003; Hu et al., BMC Genomics 7:96, 2006), a novel subtype named “Immuno” was identified by using the methods and index of the invention. EPIG program (Chou J W et al. BMC Bioinformatics 8:427, 2007 and Zhou T et al. Environmental Health Perspectives 114: 553-559) was used to identify about 200 genes closely related with immunology in tumor as demonstrated by the lowest p values. The gene list was reduced to 17 genes (designated as Immune index) by ten-fold cross validation PAM methodology. We also identified 44 of the 50 genes in PAM50 and all 21 genes in Ocotype were available on merged Affymetrix data (n=2034). A total of 66 genes (44PAM50+5 unique Oncotype+17 Immno) and 8 housekeeper genes were retrieved from 2034 patient dataset. A subset of 404 patients was identified as training dataset through ranking of one-minus-Spearman-correlation as the distance function as described (Fan et al. N. Engl. J. Med. 355:560-69, 2006). Previous work identified at least five major subtypes of breast cancer that are of prognostic and predictive value, namely Luminal A, Luminal B, Basal-like, HER2 and Normal-like (Perou et al. Nature Nature 406: 747-52,200; Sorlie T et al. Proc Natl Acad Sci USA 100: 8418-23, 2003; Hu Z et al. BMC Genomics 7: 96, 2006). Subtype classification of the tumors and the centroid predictor described (Fan et al. (N. Engl. J. Med. 355:560-69, 2006) showed statistically significant outcome predictions on the training data set. Identified in accordance with the invention was a novel subtype “Immuno” which demonstrated significant higher expression of the seventeen immune-related genes and relatively lower gene expression of the other forty nine genes with lowest expression of Basal cluster genes (FIG. 1A). The classification of six subtypes (namely Luminal A, Luminal B, Basal-like, HER2 and Normal-like and including a novel “Immuno” subtype) in breast cancer was named as “IRDM subtype” herein. IRDM subtype identified 1865 (96%) (Table 1) with accurate subtypes of total 1951 samples that had complete clinical data including particularly node status and tumor size. However, the PAM50 only classified 1697 (87%) (Table 1) with accurate subtypes of those 1951 samples. In IRDM subtyping algorithm, also low confidence cases (confidence <95%) were classified into “Mixed”, and not include in the subsequent analysis. Hence, IRDM subtype significantly advanced the utility and accuracy of breast cancer subtyping. Six IRDM subtypes demonstrated stronger outcome prediction with significant p value (p value=1.2E-21) within 1865 classified patients (FIG. 1B). As discovered, immuno subtype demonstrated a medium outcome while the Basal-like, HER2, and Luminal B performed worst in 10 year follow up (FIG. 1B). IRDM subtype prognosis value was validated in different dataset such as NKI337 done with two-color microarray (p value=2.7E-10) (Chang et al., Proc. Natl. Acad. Sci. USA 102:3738-43, 2005) and TCGA RNA-seq (TCGA. Nature 490: 61-70, 2012) done with Illumina NGS platform (p value=9E-3).

FIG. 1. Discovery of a novel subtype “Immuno” and its performance in breast cancer prognosis. Western1951 included 1951 merged breast cancer patients from original 14 public cohorts. A. Clustering heatmap of training dataset (N=404) derived from Western1951 using 66 genes in iRDM six subtype classification. B. Kaplan-Meier plot of Distance Metastasis Free Survival (DMFS) for Western patients with iRDM six subtypes (N=1865, P=1.2E-21) excluding 86 Notype patients. C. Kaplan-Meier plot of DMFS for Western patients with PAM50 five subtypes (N=1697, P=2.2E-22) excluding 254 Notype patients.

TABLE 1
Comparison of IRDM and PAM50 for breast cancer subtyping. Western patients included
1951 merged samples from 14 public cohorts. Classified percentage excluded “Mixed”
subtypes that were unable to be determined accurately based on confidence (<95%).
Immune groups, iweak and istrong, were also counted in IRDM method.

| | | | | | | | Normal Mixed | Western |
|---|---|---|---|---|---|---|---|---|
| Data Set | Methods | Basal | HER2 | Immuno | LumA | LumB | Classified (%) | (n = 1951) |
| iRDM | 310 | 209 | 343 | 443 | 351 | 209 | 86 | 96 |
| iweak | 146 | 79 | 0 | 351 | 314 | 71 | 56 | |
| istrong | 164 | 130 | 343 | 92 | 37 | 138 | 30 | |
| PAM50 | 360 | 253 | 0 | 483 | 363 | 238 | 254 | 87 |

Selection of Prediction Models

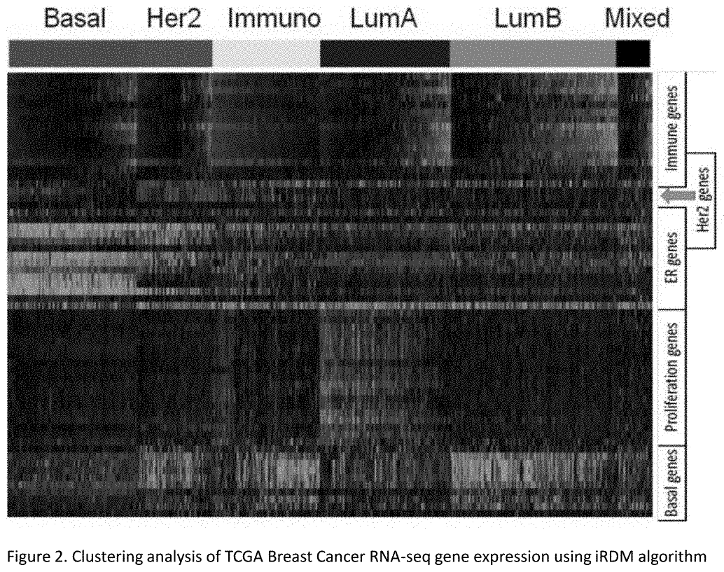

The progression of a tumor from localized, to a regional metastasis, and ultimately into a distant metastasis is hypothesized to be reflected by changes in gene and protein expression. To demonstrate the utility of a panel comprised of a plurality of the immune-related genes (comprised of the seventeen immune-related genes of the invention), analysis of Affymetrix gene expression data on selected 1681 patients (all have clinical data including tumor size) was performed using X tile program which is available through Yale (Camp et al., Clin. Cancer Res. 10:7252-59, 2004). Four models for analysis of Risk of Distance Metastasis (RDM) were developed. The models included six IRDM subtypes, proliferation score, and clinical tumor size in different combination. The model RDM-PT showed the best relative risk in the three groups (high, mediate and low risk) and lowest p value (Table 2). Proliferation and tumor size synergic boost p value over 100 folds. Thus, RDM-PTwas chosen as the outcome prediction model for further analysis. We also observed RDM-PT had the best relative risk among the three risk groups (Table 2). Immno index score was not included in the models due to their genes were already calculated in the Immuno subtype by Spearman correlation. This selected model RDM-PT was validated in different dataset such as NKI337 (Chang et al., Proc. Natl. Acad. Sci. USA 102:3738-43, 2005) done with two-color microarray (N=337, p value=7E-11) and TCGA RNA-seq (TCGA. Nature 490: 61-70, 2012) done with Illumina NGS (p value=4.4E-7). As shown in FIG. 2, immune index genes in this invention clearly recaptured a group of distinct breast tumors “Immuno” demonstrating significant higher expression of the seventeen immune index genes and relatively lower gene expression of the rest of the genes with lowest expression of Basal cluster genes (FIG. 1A). This validated the useful untility of the most advanced method Next Generation Sequencing (NGS) in tumor classification.

FIG. 2. Clustering analysis of TCGA Breast Cancer RNA-seq gene expression using subtype classification genes. The immune index 17 genes were included to classify breast cancer into five subtypes (Normal were excluded) in the TCGA data set (N=951, Nature 490: 61-70, 2012). With each of the five subtypes, tumor samples were order decreasingly by the immune index gene expression values. Mixed were those tumor samples whose confidence were less than 95% and may represent a small group of tumors without distinct gene expression pattern compared to classified subtypes.

TABLE 2
Comparison of four models for Risk of Distance Metastasis (RDM) outcome prediction
based on IRDM subtypes. X tile program was used to calculate relative risk
and the cutoff RDM score were at 40 and 60. “S” represents subtype
only, “T” tumor size and “P” proliferation score in the names of four models.

| X-Tile (n = 1681) | RDM-S | RDM-P | RDM-T | RDM-PT |
|---|---|---|---|---|
| Chi-sq Hi/Med/Low | 141 | 140 | 144 | 151 |
| P value | 8.659E−31 | 3.920E−31 | 3.341E−32 | 2.910E−33 |
| Hi (RDM score > 60) | 665 | 661 | 618 | 631 |
| Med (RDM score > 40 & < 60) | 394 | 402 | 415 | 410 |
| Low (RDM score < 40) | 622 | 618 | 648 | 640 |
| Hi (% of total patients) | 40 | 39 | 37 | 38 |
| Med (% of total patients) | 23 | 24 | 25 | 24 |
| Low (% of total patients) | 37 | 37 | 39 | 38 |
| Relative Risk Low vs Med vs Hi | 1.0/1.6/2.9 | 1.0/1.5/2.8 | 1.0/2.1/3.2 | 1.0/2.1/3.3 |

Next, we performed a similar data analysis for the same dataset using method similar to Oncotype (Paik et al., N. Engl. J. Med. 351:2817-26, 2004). The 5 housekeeper genes were used to calibrate gene expression data and the 16 genes were used to calculate RDM21 scores (Risk of Distance Metastasis using Oncotype 21 genes). Four models were evaluated using X tile program. Immuno scores alone boosted p values about 100 folds (Table 3). Both tumor size and Immuno scores had significant synergic impact on survival analysis as reflected in P values (10000 folds) and relative risk among the three risk groups. This selected model RDM21-IT was validated in different dataset such as NK1337 done with two-color microarray (p value=4.3E-11) and TCGA RNA-seq done with Illumina NGS (p value=3.8E-6). By evaluating the distribution of patients in each group in RDM-PT (Table 1) and RDM21-IT (Table 3), it was found that RDM-PT can differentiate more patients in mediate (Med) (24% RDM-PT versus 34% RDM21-IT) risk group to low risk group.

TABLE 3
Comparison of four models for Risk of Distance Metastasis prediction using
algorithm similar to Oncotype. X tile program was used to calculate relative
risk and the cutoff RDM21 score were at 40 and 60. All RDM21 scores were
scaled from 0 (lowest risk) to 100 (highest risk). “I” for Immune
index. “T” for tumor size. P values were from Kaplan-Meier survival
plot of three risk groups defined by each model.

| X-Tile (n = 1681) | RDM21 | RDM21-I | RDM21-T | RDM21-IT |
|---|---|---|---|---|
| Chi-square Hi/Med/Low | 150 | 157 | 164 | 167 |
| P value | 2.217E−33 | 1.977E−35 | 4.199E−36 | 4.920E−37 |
| Hi (RDM21 score > 60) | 586 | 558 | 490 | 468 |
| Med (RDM21 score > 40 & < 60) | 490 | 515 | 569 | 579 |
| Low (RDM21 score < 40) | 605 | 608 | 622 | 634 |
| Hi (% of total patients) | 35 | 33 | 29 | 28 |
| Med (% of total patients) | 29 | 31 | 34 | 34 |
| Low (% of total patients) | 36 | 36 | 37 | 38 |
| Relative Risk Low vs Med vs Hi | 1.0/2.3/3.6 | 1.0/2.0/3.6 | 1.0/2.3/4.2 | 1.0/2.5/4.4 |

Outcome Prediction of Immune Index Alone

The identified immune index contained seventeen genes that were closely related to tumor immunity based on EPIG result. As a first step in the evaluation of the immune index genes, we created immune index which is an average expression ratio for each patient across all seventeen genes and then looked at correlations with clinical outcome. By dividing the patients into two groups, iweak (immune index value <0) and istrong (immune index value >0) using cutoffs determined and optimized by the program X-tile (Camp et al., Clin. Cancer Res. 10:7252-59, 2004), it was determined that the immune index was prognostic of DMFS (Distant Metastasis Free Survival) with the higher gene expression portending a good or better outcome. The seventeen immune index predicts all patients outcome significantly (p value=0.0009) in the merged Affymetrix dataset (see examples in Table 4).

TABLE 4
Immune index and patient clinical outcomes. Immune index were calculated
as described in mthod section of this patent and 28 patients' clinical
data were listed as examples along with immune index and the immuno group
(iweak and istrong) based on the cut off value at 0 optimized by X-tile.

| | Immune | | DMFS TIME | DMFS | ER | PgR |
|---|---|---|---|---|---|---|
| Sample Name | index | Group | (Years) | EVENT | status | status |
| GEO\|GSE3494\|GSM79325 | 1.3 | istrong | 9.1 | 1 | ER+ | PgR− |
| GEO\|GSE2034\|GSM36983 | 0.9 | istrong | 6.3 | 1 | ER+ | NA |
| GEO\|GSE2034\|GSM37013 | 0.7 | istrong | 5.5 | 1 | ER+ | NA |
| GEO\|GSE11121\|GSM282412 | 0.2 | istrong | 9.3 | 1 | NA | NA |
| GEO\|GSE11121\|GSM282437 | 1.5 | istrong | 9.8 | 0 | NA | NA |
| GEO\|GSE11121\|GSM282380 | 1.3 | istrong | 7.1 | 0 | NA | NA |
| GEO\|GSE3494\|GSM79364 | 1.2 | istrong | 10.0 | 0 | ER+ | PgR+ |
| GEO\|GSE2034\|GSM36817 | 1.2 | istrong | 9.4 | 0 | ER+ | NA |
| GEO\|GSE3494\|GSM79216 | 1.1 | istrong | 10.0 | 0 | ER+ | PgR+ |
| GEO\|GSE7390\|GSM178076 | 0.9 | istrong | 10.0 | 0 | ER+ | NA |
| GEO\|GSE6532\|GSM151341 | 0.8 | istrong | 8.8 | 0 | ER+ | PgR− |
| GEO\|GSE2034\|GSM36932 | 0.6 | istrong | 8.0 | 0 | ER+ | NA |
| GEO\|GSE3494\|GSM79316 | 0.4 | istrong | 9.9 | 0 | ER+ | PgR+ |
| GEO\|GSE2034\|GSM37016 | 0.3 | istrong | 9.0 | 0 | ER− | NA |
| GEO\|GSE9195\|GSM232226 | 0.1 | istrong | 8.4 | 0 | ER+ | PgR− |
| GEO\|GSE1456\|GSM107166 | −0.2 | iweak | 6.2 | 1 | ER− | PgR− |
| GEO\|GSE2034\|GSM36778 | −0.4 | iweak | 4.2 | 1 | ER+ | NA |
| GEO\|GSE2603\|GSM50062 | −0.9 | iweak | 3.1 | 1 | ER+ | PgR+ |
| GEO\|GSE3494\|GSM79321 | −1.1 | iweak | 0.0 | 1 | ER+ | PgR+ |
| GEO\|GSE11121\|GSM282485 | −0.1 | iweak | 3.9 | 0 | NA | NA |
| GEO\|GSE6532\|GSM65362 | −0.2 | iweak | 7.6 | 0 | ER+ | NA |
| GEO\|GSE1456\|GSM107132 | −0.2 | iweak | 7.3 | 0 | ER+ | PgR+ |
| GEO\|GSE1456\|GSM107218 | −0.4 | iweak | 6.4 | 0 | ER− | PgR− |
| GEO\|GSE2034\|GSM36785 | −0.4 | iweak | 4.8 | 0 | ER+ | NA |
| GEO\|GSE2603\|GSM50037 | −0.5 | iweak | 6.9 | 0 | ER+ | PgR+ |
| GEO\|GSE9195\|GSM232241 | −0.7 | iweak | 7.2 | 0 | ER+ | PgR+ |
| GEO\|GSE11121\|GSM282419 | −0.9 | iweak | 6.2 | 0 | NA | NA |
| GEO\|GSE3494\|GSM79211 | −1.1 | iweak | 0.1 | 0 | ER+ | PgR+ |

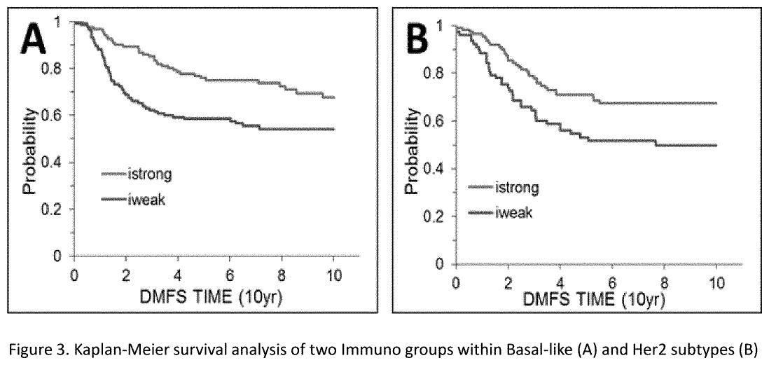

FIG. 3. Kaplan-Meier survival analysis of two Immuno groups (istrong and iweak) for patients within Basal-like subtype (panel A, N=310, P=0.002) and Her2 subtype (panel B, N=209, P=0.011) in the merged Affymetrix dataset Western1951 (N=1951).

Applying the immune index classification rules to each subtype revealed only within either Basal-like subtype (FIG. 3A) or Her2 subtype (FIG. 3B) significantly predicted outcomes. Better overall immune index outcome prediction was contributed mainly by Basal-like and Her2 tumors. In order to evaluate the minimum genes required to achieve statistical significance in outcome prediction, we randomly reiterated a maximum 1000 times and calculated Kaplan-Meier survival analysis P values separately by picking 5, 6, 7, 8, 9, 10, 11, 12, 13, 14, 15, 16 genes of the seventeen “immune index” genes (Table 5). All combination of genes as showed in the Table 4 were significant (maximum P value <5.0E-2). This result provided strong evidence to support that the methods and index of the invention can use at least six biomarkers selected from the immune-related group consisting of APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A, with six as the minimum gene number in a panel comprising a plurality of immune-related genes.

TABLE 5
Kaplan-Meier survival analysis P values for using
5, 6, 7, 8, 9, 10, 11, 12, 13, 14, 15, 16 genes of the
seventeen immune-related genes separately. Each
number of genes was picked randomly and
reiterated 1000 times to obtain maximum P value
and minimum P value.

| P | 5 | 6 | 7 | 8 | 9 | 10 |
|---|---|---|---|---|---|---|
| value | genes | genes | genes | genes | genes | genes |
| maxi- | 1.4E−02 | 8.4E−03 | 1.4E−03 | 2.5E−03 | 1.4E−03 | 8.6E−04 |
| mum | | | | | | |
| mini- | 7.3E−19 | 2.8E−16 | 7.8E−15 | 3.6E−13 | 4.0E−13 | 1.4E−14 |
| mum | | | | | | |
| P | 11 | 12 | 13 | 14 | 15 | 16 |
| value | genes | genes | genes | genes | genes | genes |
| maxi- | 2.9E−04 | 2.2E−04 | 1.1E−04 | 5.5E−05 | 3.2E−05 | 1.0E−05 |
| mum | | | | | | |
| mini- | 2.4E−11 | 2.3E−11 | 2.7E−10 | 5.5E−05 | 3.2E−05 | 1.0E−05 |
| mum | | | | | | |

Next we determined which risk group was affected by immune index. Immune index predicted outcome only in high risk group identified by both RDM-PT (p value=3E-5) and RDM21-IT (p value=7E-3). The outcome prediction or prognosis value of the immune index in high risk group was also validated in independent dataset across different platforms such as NKI 337 (Chang et al., Proc. Natl. Acad. Sci. USA 102:3738-43, 2005)(N=337, p value=0.04) performed on two color Agilent microarrays and TCGA breast cancer RNA-seq (N=951, p value=0.05)(TCGA. Nature 490: 61-70, 2012).

Although the foregoing invention has been described in some detail by way of illustration and example for purposes of clarity of understanding, it will be obvious that certain changes and modifications may be practiced within the scope of the appended claims.

What is claimed is:

1. A method for evaluating the prognosis of a cancer patient, comprising
(a) determining expression levels of at least six biomarkers selected from the group consisting of APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A (SEQ ID NOs: 1-17) in a biological sample co from said patient,
(b) normalizing the expression levels from step (a) against the expression levels of RNA transcripts or their expression products in said sample, or comparing the expression levels from step (a) with a reference set of expression levels for the biomarkers derived from healthy individuals; wherein expression of said biomarkers in a higher amount (overexpression) from step (a) is an indicator of prognosis.

2. The method of claim 1, wherein overexpression of said biomarkers is indicative of a good or better prognosis.

3. The method of claim 1, wherein absence of overexpression of said biomarkers is indicative of a bad or worse prognosis.

4. The method of claim 1, wherein measuring gene expression of said biomarkers includes performing nucleic acid hybridization, quantitative RT-PCR, NGS, immunohistochemistry or any other techniques.

5. The method of claim 1, wherein said method for evaluating the prognosis of a breast cancer patient further comprises assessment of clinical information.

6. The method of claim 5, wherein said clinical information comprises tumor size, tumor grade, lymph node status, and family history.

7. The method of claim 6, wherein said method is used to develop a treatment strategy for said breast cancer patient.

8. The method of claim 1, wherein said method for evaluating the prognosis of a breast cancer patient is coupled with analysis of other biomarker such as ER, PR, or Her-2 expression levels and other diagnosis tests.

9. The method of claim 1, wherein said method for evaluating the prognosis of a breast cancer patient is independent of estrogen receptor status of said patient.

10. The method of claim 1, wherein said method is used to evaluate the prognosis of an estrogen receptor-positive or an estrogen receptor-negative breast cancer patient.

11. The method of claim 1, wherein said RNA is isolated from a fixed, paraffin-embedded sample comprising one or more than one cancer cells from said patient.

12. The method of claim 1, wherein said RNA is isolated from core biopsy tissue or fine needle aspirate cells comprising one or more than one cancer cell from said patient.

13. The method of claim 1, wherein the levels of expression are converted into an immuno index, and the immune index is an indicator for prognosis.

14. A method for evaluating the prognosis of a cancer patient, comprising determining the expression levels of the RNA transcripts of at least six biomarkers selected from the group consisting of APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A (SeQ ID NOs: 1-17) in a biological sample from said patient, normalized against the expression levels of all RNA transcripts in said sample, wherein overexpression of said biomarkers is indicative of a good or better prognosis as compared to absence of overexpression in a cancer patient.

15. A method for evaluating the prognosis of a breast cancer patient, comprising determining the expression levels from the group consisting of APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A in a sample comprising one or more than one cancer cell from said patient, normalized against the expression levels of a reference set of RNA transcripts in said sample, wherein overexpression of said biomarkers is indicative of a good or better prognosis, thereby evaluating the prognosis of said breast cancer patient.

16. A method for predicting a response of a breast cancer patient to a selected treatment, comprising determining the expression levels of the RNA transcripts or their expression products of at least six biomarkers selected from the group consisting of APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A (SEQ ID NOs: 1-17) in a sample comprising a cancer cell from said patient, normalized against the expression levels of all RNA transcripts or their expression products in said sample, or of a reference set of RNA transcripts or their expression products in said sample, wherein overexpression of said biomarkers is indicative of a positive treatment response.

17. The method of claim 16, wherein said treatment comprises gene therapy or immunotherapy.

18. The method of claim 17, wherein said immunotherapy comprises a monoclonal antibody.

19. A method for evaluating the prognosis of a breast cancer patient, comprising detecting expression of at least six biomarkers selected from the group consisting of APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A (SEQ ID NOs:1-17) in a sample from said patient, wherein overexpression of said biomarkers is indicative of a good or better prognosis.

20. A kit comprising of nucleic acid probes for at least 6 of the immune-related genes selected from the group consisting of APOBEC3G, CCL5, CCR2, CD2, CD27, CD3D, CD52, CORO1A, CXCL9, GZMA, GZMK, HLA-DMA, IL2RG, LCK, PRKCB, PTPRC, SH2D1A (see SEQ ID NOs: 18-34 as examples, not limited to those listed probes).


SEQUENCE LISTING (34 sequences)

SEQ ID NO: 1 - DNA, 1848 bp, Homo Sapiens
  mRNA (1)..(1848): gi|304282223|ref|NM_021822.3| Homo sapiens apolipoprotein B mRNA editing enzyme, catalytic polypeptide-like 3G (APOBEC3G), mRNA
gtgctctgctggctcagcctggtgtggacccacctcccgggcgctggctgcaatgactttctctttccctttgcaattgccttgggtcctgccgcacagagcggcctgtctttatcagaggtccctctgccagggggagggccccagagaaaaccagaaagagggtgagagactgaggaagataaagcgtcccagggcctcctacaccagcgcctgagcaggaagcgggaggggccatgactacgaggccctgggaggtcactttagggagggctgtcctaaaaccagaagcttggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatctttgttgcccgcctctactacttctgggacccagattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactgaaggatgggcctcagtctctaaggaaggcagagacctgggttgagcctcagaataaaagatcttcttccaagaaatgcaaacaggctgttcaccaccatctccagctgatcacagacaccagcaaagcaatgcactcctgaccaagtagattcttttaaaaattagagtgcattactttgaatcaaaaatttatttatatttcaagaataaagtactaagattgtgctcaatacacagaaaagtttcaaacctactaatccagcgacaatttgaatcggttttgtaggtagaggaataaaatgaaatactaaatctttctgtaaaaaaaaaaaaaaa

SEQ ID NO: 2 - DNA, 1319 bp, Homo Sapiens
  mRNA (1)..(1319): Homo sapiens chemokine (C-C motif) ligand 5 (CCL5)
gctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtccacaggtcaaggatgccaaagagagagggacagcaagtctggcaggatttcctgtatgactcccggctgaacaagggcaagctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctaccacacagcagcagttacaaaaaccttccccaggctggacgtggtggctcacgcctgtaatcccagcactttgggaggccaaggtgggtggatcacttgaggtcaggagttcgagaccagcctggccaacatgatgaaaccccatctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgccagggctctgtgaccaggaaggaagtcagcatgcctctagaggcaaggaggggaggaacactgcactcttaagcttccgccgtctcaacccctcacaggagcttactggcaaacatgaaaaatcggcttaccattaaagttctcaatgcaaccataaaaaaaaaa

SEQ ID NO: 3 - DNA, 1113 bp, Homo Sapiens
  mRNA (1)..(1113): Homo Sapiens Chemokine (C-C motif) receptor 2 (CCR2); GenBank Accession No. NM_001194959
atggatggcaatgatacattcagtcacaatgtgcttcccacatctcactctctgtttacaacaaatgtcaaggggaatgatgaagaacccaccaccagttatgactatgattacagtgaaccctgccgaaagaccagcgtgggacaaatcgaagcacagctcctgccgccgctctactcgctggtcttcatctttggttttgtgggcaacctgctggttgtccttatcctaatcaactgcaaaaagctgaagagcatgactgacatctacctgcttaacttggccatctctgacctgctgttcctcctcaccatgccgttctgggctcactatgctgcagaccagtgggtttttgggaatgtgatgtgcaaatttttcacagggctgtatcacattggttattttggtggaatcttcttcatcatccttttgacaatcgataggtacctggctattgtccatgctgtgtttgctttaaaagccaggacagtcacctttggggtggtgacaagtggggtcacctgggtggtggctgtgtttgcctctctcccgggaatcatctttatcaaatccctcgaagaacattcaggttatgcctgtgccccttattttccactaggatggaagaatttccatacaattatgaggagcatcttggggctggtgctgccactgcttgtcatgatcatctgctactcaggaatcataaaaaccctgctccggtgtcgcaatgagaagaagaagcacaaggctgtgaggctcatcttcgtgatcatgattgtctactttctcttctgggctccctacaacatcgtccttctcctgagcaccttccaggaattctttggcttgagtaactgtaagagcagcagtcagctggaccaagccatgcaggtgacagagaccctggggctgacccactgctgcatcaaccccatcatctacgcctttgttggggagaagttcaggaggtatctctccacgttcttccgaaagcatattgccaaacacctctgcaaacaatgcccagttttctatggggagacaggagatcgagtgagttcaacatacacccattctactggggaacaggaagtctcagctgctttatag

SEQ ID NO: 4 - DNA, 1595 bp, Homo Sapiens
  mRNA (1)..(1595): gi|156071471|ref|NM_001767.3| Homo sapiens CD2 molecule (CD2), mRNA
agaatcaaaagaggaaaccaacccctaagatgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtccagagaaaggtctggacatctatctcatcattggcatatgtggaggaggcagcctcttgatggtctttgtggcactgctcgttttctatatcaccaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaaaaaagatagaaactgtctttttcaataaaaagcactgtggatttctgccctcctgatgtgcatatccgtacttccatgaggtgttttctgtgtgcagaacattgtcacctcctgaggctgtgggccacagccacctctgcatcttcgaactcagccatgtggtcaacatctggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgcttaagagactctggagtttcttatgtgccctggtggacacttgcccaccatcctgtgagtaaaagtgaaataaaagctttgactagaaaaaaaaaaaaaaaaaaaaaaaaaaaaaaaaaaaaaaaaaaaaaaaaaaaaaaaaaaaaaaaaa

SEQ ID NO: 5 - DNA, 1320 bp, Homo Sapiens
  mRNA (1)..(1320): gi|117422442|ref|NM_001242.4| Homo sapiens CD27 molecule (CD27), mRNA
cggaaggggaagggggtggaggttgctgctatgagagagaaaaaaaaaacagccacaatagagattctgccttcaaaggttggcttgccacctgaagcagccactgcccagggggtgcaaagaagagacagcagcgcccagcttggaggtgctaactccagaggccagcatcagcaactgggcacagaaaggagccgcctgggcagggaccatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctgagccagcacctgcgggagctgcactacagccctggcctccacccccaccccgccgaccatccaagggagagtgagacctggcagccacaactgcagtcccatcctcttgtcagggccctttcctgtgtacacgtgacagagtgccttttcgagactggcagggacgaggacaaatatggatgaggtggagagtgggaagcaggagcccagccagctgcgcctgcgctgcaggagggcgggggctctggttgtaaaacacacttcctgctgcgaaagacccacatgctacaagacgggcaaaataaagtgacagatgaccaccctgca

SEQ ID NO: 6 - DNA, 771 bp, Homo Sapiens
  mRNA (1)..(771): gi|98985799|ref|NM_000732.4| Homo sapiens CD3d molecule, delta (CD3-TCR complex) (CD3D)
agagaagcagacatcttctagttcctcccccactctcctctttccggtacctgtgagtcagctaggggagggcagctctcacccaggctgatagttcggtgacctggctttatctactggatgagttccgctgggagatggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtgaacctgagactggtggcttctagaagcagccattaccaactgtaccttcccttcttgctcagccaataaatatatcctctttcactcagaaaaaaaaaaaaaaaaaaaaaaaaaaaaaa

SEQ ID NO: 7 - DNA, 523 bp, Homo Sapiens
  mRNA (1)..(523): gi|68342029|ref|NM_001803.2| Homo sapiens CD52 molecule (CD52), mRNA
ctcctggttcaaaagcagctaaaccaaaagaagcctccagacagccctgagatcacctaaaaagctgctaccaagacagccacgaagatcctaccaaaatgaagcgcttcctcttcctcctactcaccatcagcctcctggttatggtacagatacaaactggactctcaggacaaaacgacaccagccaaaccagcagcccctcagcatccagcaacataagcggaggcattttccttttcttcgtggccaatgccataatccacctcttctgcttcagttgaggtgacacgtctcagccttagccctgtgccccctgaaacagctgccaccatcactcgcaagagaatcccctccatctttgggaggggttgatgccagacatcaccaggttgtagaagttgacaggcagtgccatgggggcaacagccaaaataggggggtaatgatgtaggggccaagcagtgcccagctgggggtcaataaagttacccttgtacttgcaaaaaaaaaaaaaaaaaaa

SEQ ID NO: 8 - DNA, 1943 bp, Homo Sapiens
  mRNA (1)..(1943): gi|306482594|ref|NM_001193333.2| Homo sapiens coronin, actin binding protein, 1A (CORO1A), transcript variant 1, mRNA
aaaaagaagtttagattcgcttcccggagccgggatggcagcctgcgctatggttggggaccaacgtggtgcacgggcaggggccggggagagaggcggccgcagcgggagcccgggagcgcagggcgggcctggaagagctccgccccaaggggcgcggccaccccggaggcgggcgcacggctgcttctcattcattgtcttgacaagagcatcttcagcgggcgagtccccggctcctccagctccttcctcctcttcctcctcctcctccacctccggcttttgggggatcactgtcctctctcggcagcagatacaggctgctcctcaagactggggctcttcacatacgctcctcaccgctctttgggcaggccgacctagcagcctcttcgggagtcctggggtggccgggggtgctggagcccccaaatgagccggcaggtggtccgctccagcaagttccgccacgtgtttggacagccggccaaggccgaccagtgctatgaagatgtgcgcgtctcacagaccacctgggacagtggcttctgtgctgtcaaccctaagtttgtggccctgatctgtgaggccagcgggggaggggccttcctggtgctgcccctgggcaagactggacgtgtggacaagaatgcgcccacggtctgtggccacacagcccctgtgctagacatcgcctggtgcccgcacaatgacaacgtcattgccagtggctccgaggactgcacagtcatggtgtgggagatcccagatgggggcctgatgctgcccctgcgggagcccgtcgtcaccctggagggccacaccaagcgtgtgggcattgtggcctggcacaccacagcccagaacgtgctgctcagtgcaggttgtgacaacgtgatcatggtgtgggacgtgggcactggggcggccatgctgacactgggcccagaggtgcacccagacacgatctacagtgtggactggagccgagatggaggcctcatttgtacctcctgccgtgacaagcgcgtgcgcatcatcgagccccgcaaaggcactgtcgtagctgagaaggaccgtccccacgaggggacccggcccgtgcgtgcagtgttcgtgtcggaggggaagatcctgaccacgggcttcagccgcatgagtgagcggcaggtggcgctgtgggacacaaagcacctggaggagccgctgtccctgcaggagctggacaccagcagcggtgtcctgctgcccttctttgaccctgacaccaacatcgtctacctctgtggcaagggtgacagctcaatccggtactttgagatcacttccgaggcccctttcctgcactatctctccatgttcagttccaaggagtcccagcggggcatgggctacatgcccaaacgtggcctggaggtgaacaagtgtgagatcgccaggttctacaagctgcacgagcggaggtgtgagcccattgccatgacagtgcctcgaaagtcggacctgttccaggaggacctgtacccacccaccgcagggcccgaccctgccctcacggctgaggagtggctggggggtcgggatgctgggcccctcctcatctccctcaaggatggctacgtacccccaaagagccgggagctgagggtcaaccggggcctggacaccgggcgcaggagggcagcaccagaggccagtggcactcccagctcggatgccgtgtctcggctggaggaggagatgcggaagctccaggccacggtgcaggagctccagaagcgcttggacaggctggaggagacagtccaggccaagtagagccccgcagggcctccagcagggtcagccattcacacccatccactcacctcccattcccagccacatggcagagaaaaaaatcataataaaatggctttattttctggtaaaaaaaaaaa

SEQ ID NO: 9 - DNA, 2723 bp, Homo Sapiens
  mRNA (1)..(2723): gi|692314725|ref|NM_002416.2| Homo sapiens chemokine (C-X-C motif) ligand 9 (CXCL9), mRNA
aaaatgtgttctctaaagaatttctcaggctcaaaatccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcagaaaacagatcttgattttttttctcttgaatgtacccttcaactgttgaatgtttaatagtaaatcttatatgtccttatttactttttagctttctctcaaataaagtgtaacactagttgagataaaaaaaaaaaaaaaaa

SEQ ID NO: 10 - DNA, 913 bp, Homo Sapiens
  mRNA (1)..(913): gi|194097328|ref|NM_006144.3| Homo sapiens granzyme A (granzyme 1, cytotoxic T-lymphocyte-associated serine esterase 3) (GZMA), mRNA
agattttcaggttgattgatgtgggacagcagccacaatgaggaactcctatagatttctggcatcctctctctcagttgtcgtttctctcctgctaattcctgaagatgtctgtgaaaaaattattggaggaaatgaagtaactcctcattcaagaccctacatggtcctacttagtcttgacagaaaaaccatctgtgctggggctttgattgcaaaagactgggtgttgactgcagctcactgtaacttgaacaaaaggtcccaggtcattcttggggctcactcaataaccagggaagagccaacaaaacagataatgcttgttaagaaagagtttccctatccatgctatgacccagccacacgcgaaggtgaccttaaacttttacagctgacggaaaaagcaaaaattaacaaatatgtgactatccttcatctacctaaaaagggggatgatgtgaaaccaggaaccatgtgccaagttgcagggtggggcaggactcacaatagtgcatcttggtccgatactctgagagaagtcaatatcaccatcatagacagaaaagtctgcaatgatcgaaatcactataattttaaccctgtgattggaatgaatatggtttgtgctggaagcctccgaggtggaagagactcgtgcaatggagattctggaagccctttgttgtgcgagggtgttttccgaggggtcacttcctttggccttgaaaataaatgcggagaccctcgtgggcctggtgtctatattcttctctcaaagaaacacctcaactggataattatgactatcaagggagcagtttaaataaccgtttcctttcatttactgtggcttcttaatcttttcacaaataaaatcaatttgcatgactgtaaaaaaaaaaaaaaaaaa

SEQ ID NO: 11 - DNA, 1074 bp, Homo Sapiens
  mRNA (1)..(1074): gi|73747815|ref|NM_002104.2| Homo sapiens granzyme K (granzyme 3; tryptase II) (GZMK), mRNA
gatcaacacatttcatctgggcttcttaaatctaaatctttaaaatgactaagttttcttccttttctctgtttttcctaatagttggggcttatatgactcatgtgtgtttcaatatggaaattattggagggaaagaagtgtcacctcattccaggccatttatggcctccatccagtatggcggacatcacgtttgtggaggtgttctgattgatccacagtgggtgctgacagcagcccactgccaatatcggtttaccaaaggccagtctcccactgtggttttaggcgcacactctctctcaaagaatgaggcctccaaacaaacactggagatcaaaaaatttataccattctcaagagttacatcagatcctcaatcaaatgatatcatgctggttaagcttcaaacagccgcaaaactcaataaacatgtcaagatgctccacataagatccaaaacctctcttagatctggaaccaaatgcaaggttactggctggggagccaccgatccagattcattaagaccttctgacaccctgcgagaagtcactgttactgtcctaagtcgaaaactttgcaacagccaaagttactacaacggcgacccttttatcaccaaagacatggtctgtgcaggagatgccaaaggccagaaggattcctgtaagggtgactcagggggccccttgatctgtaaaggtgtcttccacgctatagtctctggaggtcatgaatgtggtgttgccacaaagcctggaatctacaccctgttaaccaagaaataccagacttggatcaaaagcaaccttgtcccgcctcatacaaattaagttacaaataattttattggatgcacttgcttcttttttcctaatatgctcgcaggttagagttgggtgtaagtaaagcagagcacatatggggtccatttttgcacttgtaagtcattttattaaggaatcaagttctttttcacttgtatcactgatgtatttctaccatgctggttttattctaaataaaatttagaagactcaaaaaaaaaaaaaaaaaaaaaaaaaaaaa

SEQ ID NO: 12 - DNA, 1122 bp, Homo Sapiens
  mRNA (1)..(1122): gi|207113141|ref|NM_006120.3| Homo sapiens major histocompatibility complex, class II, DM alpha (HLA-DMA), mRNA
gatctaaggccaccctctcggggagggagttggggaagctgggttggctgggttggtagctcctacctactgtgtggcaagaaggtatgggtcatgaacagaaccaaggagctgcgctgctacagatgttaccacttctgtggctgctaccccactcctgggccgtccctgaagctcctactccaatgtggccagatgacctgcaaaaccacacattcctgcacacagtgtactgccaggatgggagtcccagtgtgggactctctgaggcctacgacgaggaccagcttttcttcttcgacttttcccagaacactcgggtgcctcgcctgcccgaatttgctgactgggctcaggaacagggagatgctcctgccattttatttgacaaagagttctgcgagtggatgatccagcaaatagggccaaaacttgatgggaaaatcccggtgtccagagggtttcctatcgctgaagtgttcacgctgaagcccctggagtttggcaagcccaacactttggtctgttttgtcagtaatctcttcccacccatgctgacagtgaactggcagcatcattccgtccctgtggaaggatttgggcctacttttgtctcagctgtcgatggactcagcttccaggccttttcttacttaaacttcacaccagaaccttctgacattttctcctgcattgtgactcacgaaattgaccgctacacagcaattgcctattgggtaccccggaacgcactgccctcagatctgctggagaatgtgctgtgtggcgtggcctttggcctgggtgtgctgggcatcatcgtgggcattgttctcatcatctacttccggaagccttgctcaggtgactgattcttccagaccagagtttgatgccagcagcttcggccatccaaacagaggatgctcagatttctcacatcctgcccaggatctcctcttagggtagaagtctctgggacatccctggggtgtgtgtgtagatttcccacctggggactctgctgtccctgggcttgcatcccagggatcccagagtggcctgcctatcacaaccacatcccttccccccacaaggcaataaatctcatttctttatatc

SEQ ID NO: 13 - DNA, 1560 bp, Homo Sapiens
  mRNA (1)..(1560): gi|291045209|ref|NM_000206.2| Homo sapiens interleukin 2 receptor, gamma (IL2RG), mRNA
agaggaaacgtgtgggtggggaggggtagtgggtgagggacccaggttcctgacacagacagactacacccagggaatgaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatcaaaaaaaaaaaaaaaaaaaaaaaaaaaaaaa

SEQ ID NO: 14 - DNA, 2102 bp, Homo Sapiens
  mRNA (1)..(2102): gi|586946350|ref|NM_001042771.2| Homo sapiens LCK proto-oncogene, Src family tyrosine kinase (LCK), transcript variant 1, mRNA
gtgtgaatttacttgtagcctgagggctcagagggagcaccggtttggagctgggaccccctattttagcttttctgtggctggtgaatggggatcccaggatctcacaatctcagggaccatgggctgtggctgcagctcacacccggaagatgactggatggaaaacatcgatgtgtgtgagaactgccattatcccatagtcccactggatggcaagggcacgctgctcatccgaaatggctctgaggtgcgggacccactggttacctacgaaggctccaatccgccggcttccccactgcaagacaacctggttatcgctctgcacagctatgagccctctcacgacggagatctgggctttgagaagggggaacagctccgcatcctggagcagagcggcgagtggtggaaggcgcagtccctgaccacgggccaggaaggcttcatccccttcaattttgtggccaaagcgaacagcctggagcccgaaccctggttcttcaagaacctgagccgcaaggacgcggagcggcagctcctggcgcccgggaacactcacggctccttcctcatccgggagagcgagagcaccgcgggatcgttttcactgtcggtccgggacttcgaccagaaccagggagaggtggtgaaacattacaagatccgtaatctggacaacggtggcttctacatctcccctcgaatcacttttcccggcctgcatgaactggtccgccattacaccaatgcttcagatgggctgtgcacacggttgagccgcccctgccagacccagaagccccagaagccgtggtgggaggacgagtgggaggttcccagggagacgctgaagctggtggagcggctgggggctggacagttcggggaggtgtggatggggtactacaacgggcacacgaaggtggcggtgaagagcctgaagcagggcagcatgtccccggacgccttcctggccgaggccaacctcatgaagcagctgcaacaccagcggctggttcggctctacgctgtggtcacccaggagcccatctacatcatcactgaatacatggagaatgggagtctagtggattttctcaagaccccttcaggcatcaagttgaccatcaacaaactcctggacatggcagcccaaattgcagaaggcatggcattcattgaagagcggaattatattcatcgtgaccttcgggctgccaacattctggtgtctgacaccctgagctgcaagattgcagactttggcctagcacgcctcattgaggacaacgagtacacagccagggagggggccaagtttcccattaagtggacagcgccagaagccattaactacgggacattcaccatcaagtcagatgtgtggtcttttgggatcctgctgacggaaattgtcacccacggccgcatcccttacccagggatgaccaacccggaggtgattcagaacctggagcgaggctaccgcatggtgcgccctgacaactgtccagaggagctgtaccaactcatgaggctgtgctggaaggagcgcccagaggaccggcccacctttgactacctgcgcagtgtgctggaggacttcttcacggccacagagggccagtaccagcctcagccttgagaggccttgagaggccctggggttctccccctttctctccagcctgacttggggagatggagttcttgtgccatagtcacatggcctatgcacatatggactctgcacatgaatcccacccacatgtgacacatatgcaccttgtgtctgtacacgtgtcctgtagttgcgtggactctgcacatgtcttgtacatgtgtagcctgtgcatgtatgtcttggacactgtacaaggtacccctttctggctctcccatttcctgagaccacagagagaggggagaagcctgggattgacagaagcttctgcccacctacttttctttcctcagatcatccagaagttcctcaagggccaggactttatctaatacctctgtgtgctcctccttggtgcctggcctggcacacatcaggagttcaataaatgtctgttgatgactgttgtaca

SEQ ID NO: 15 - DNA, 8014 bp, Homo Sapiens
  mRNA (1)..(8014): gi|197100031|ref|NM_002738.6| Homo sapiens protein kinase C, beta (PRKCB), transcript variant 2, mRNA
agctggacgagcggcagcagctgggcgagtgacagccccggctccgcgcgccgcggccgccagagccggcgcaggggaagcgcccgcggccccgggtgcagcagcggccgccgcctcccgcgcctccccggcccgcagcccgcggtcccgcggccccggggccggcacctctcgggctccggctccccgcgcgcaagatggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggggcttcgggaagcagggattccagtgccaagtttgctgctttgtggtgcacaagcggtgccatgaatttgtcacattctcctgccctggcgctgacaagggtccagcctccgatgacccccgcagcaaacacaagtttaagatccacacgtactccagccccacgttttgtgaccactgtgggtcactgctgtatggactcatccaccaggggatgaaatgtgacacctgcatgatgaatgtgcacaagcgctgcgtgatgaatgttcccagcctgtgtggcacggaccacacggagcgccgcggccgcatctacatccaggcccacatcgacagggacgtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggtttaagttactgagccaggaggaaggcgagtacttcaatgtgcctgtgccaccagaaggaagtgaggccaatgaagaactgcggcagaaatttgagagggccaagatcagtcagggaaccaaggtcccggaagaaaagacgaccaacactgtctccaaatttgacaacaatggcaacagagaccggatgaaactgaccgattttaacttcctaatggtgctggggaaaggcagctttggcaaggtcatgctttcagaacgaaaaggcacagatgagctctatgctgtgaagatcctgaagaaggacgttgtgatccaagatgatgacgtggagtgcactatggtggagaagcgggtgttggccctgcctgggaagccgcccttcctgacccagctccactcctgcttccagaccatggaccgcctgtactttgtgatggagtacgtgaatgggggcgacctcatgtatcacatccagcaagtcggccggttcaaggagccccatgctgtattttacgctgcagaaattgccatcggtctgttcttcttacagagtaagggcatcatttaccgtgacctaaaacttgacaacgtgatgctcgattctgagggacacatcaagattgccgattttggcatgtgtaaggaaaacatctgggatggggtgacaaccaagacattctgtggcactccagactacatcgcccccgagataattgcttatcagccctatgggaagtccgtggattggtgggcatttggagtcctgctgtatgaaatgttggctgggcaggcaccctttgaaggggaggatgaagatgaactcttccaatccatcatggaacacaacgtagcctatcccaagtctatgtccaaggaagctgtggccatctgcaaagggctgatgaccaaacacccaggcaaacgtctgggttgtggacctgaaggcgaacgtgatatcaaagagcatgcatttttccggtatattgattgggagaaacttgaacgcaaagagatccagcccccttataagccaaaagcttgtgggcgaaatgctgaaaacttcgaccgatttttcacccgccatccaccagtcctaacacctcccgaccaggaagtcatcaggaatattgaccaatcagaattcgaaggattttcctttgttaactctgaatttttaaaacccgaagtcaagagctaagtagatgtgtagatctccgtccttcatttctgtcattcaagctcaacggctattgtggtgacatttttatgtttttcattgccaagttgcatccatgtttgattttctgatgagactagagtgacagtgtttcagaacccaaatgtcctcaggtagtttggagcatctctatgagatgggattatgcagatggcctatggaaaatgcagctgcataattaacacattatcaaagtcctcttacaatttattttccgcagcatgtcagctaagtagacccaatggggagagaaaatgcctgctttctttccctctttttctgcactgccatattcacccccaaccatccaatctgtggataattggatgttagcggtactcttccacttccgggcctggagcttggcttgtatccaagtgtatggttgctttgcctaagaggaatccctctatttcacctgttctggaggcaccagaccttgaaaagaacatgctcaaaataaaatgttatctgttatttttgtaaactcaaagttaagatgatcaaagttctaaaattccaagaatgtgcttttagacggtctcaatctaaaagcacttcaaggggtcaaagggcaaccagcttgggtgctacctcagtgttgtagtttctgatactttatgtctttgctcaccctcatccccaaactacttgaaaagggcatttggcaccactctctgaaacaacacagtcactctagcaaggcccccaaagggccctggttttacattacatttcaaactttatttgctttggggttttgtttctgttgttgttcaaatgcaaaaaaaagaaaaaaaaagaaaaaaaaaggtgactcacattgttacacatgctttaaaatatgtattcaaatgttattaaccacaatgacgacctgctttgatttaaccaagaagacggctgcggagcctagcagactcaggcctgtgggaatgggatttgttacaaatctaggtttgttactggcttcagaaagctaattaagtgctctgaaaaagacaccgtttcttgaaacaaagatggttgtattcctcactttgatgttgttttgcaagatgtttgtggaaatgttcatttgtatctggatctctgttatgtgccatttttcttctagcatcgagatacaataaaaaaaaaaaaaaagaaaagaagaagaaatactatttcaaggaaaactgctctttttgagaaacgtggacctaaactacaaagtgggaactgaggagggaactcaggagaaaggaactaactgcggagctttaatcttggccccagtgttcagccactcggaggggcgggggctgtggcccattcaggggctgctggtgggctgtagtggggtgggatgacctggccagagccaacgaggatactggagcccaaagtcaagtttagagaccagctgggaacgtgaatggggctcttgattttcttatcaaaatcaccactcctcccagcttggactaaatattctttctagcaagcagctttgtgagctccctgaagcccaaggaaacccttcggtgggagaaatttcatttctgtctgagaggattaaggcagcaggtgactccccctcctcgcctgccgtgtcctgctattctcaggcagctctaaggagaattcttatcacagttcaagtgatttccagaagttccagggcttctgagagaccatcaagggaactttaacaacttgacaaatgtccttgaagtaagatgcctcatctttagggaaaaatggggtttggatttctgcttaggcaaagtctcctgcagttcatccttctctgtcctcttcttgcttcaggcttggggaccgtccctgctgtccccactgtggtggcaatcaggacctaaggtgaagcaaacttgaagttctatctgacaagtttaggcagtaagagaaggagggaaatcggagcaaagctccctcactttattgttgagaaactggcatctggaaagaggaaggaatttgcccaaagtcagtcagctgggataaaaacctgggtgtcctgtccagaaagtgcagggtgctttctgctctgtagcaaggcagcagacatctctgagccaggcccaccaacaggcccttatctggtggttggatcatgatcccattttgcttggacatgctctcaggaagataaaaaccatggagaaacactaggccattgacaaatgatctgagacaactttagaaaacaatgtaggatgaatggaaagagaaagaaaggaaagaaagaagaaaaagaaagaaggaaagaaagaaagagaaaggaaggaaggaaagaaggaaggaaaagaaggaaggaaggaaggaatatagtgttataaatactgcactcaacattttccaaattcttgccattatttttcaaaagtttaatagtttgcagaaatagatactcaagccaaagtctgttttagagaaactttccatggaaagtcagaatttctaccacttccttttctatccacatttccagtgcagaagaaactgagaaacagagctttttgaagagaggacagggccatagcaacaaggaccttcttgggggattaatgggaggtcagtagaattaataaccctccttggatgagtgctactgttttcacatggcttcagatgctatcaacctcaaagaaatgatctcaacagagaagcttattctctcccaacttctacggtaaaatccaggagtattttctctggggatctgcccacaggacaaagtccataaaagcaagtcctgtctggaccatgtggttatctgaagcattagccatcaccagcacaacaaacggggcagggctttccaaggtggggctggtcagaagggaatctttgataagaggcccacaggcagggaaagcgaaatagggttgatgagaccaggggagacctaaaaaaaaggcagctttgtgtcttctagctccaaatatacctgccttttagctcacacactgtcctggagttctcagacctttaggggccctaacacagttcagttcatacaggggttcaaaagggacagtggcccatttgggagacctttaggatcaatgggaatcaattccattgttttgcctcagagtaaagtttctggctcggggacaattataagttgcaaaaaggatagaggcatatcccaagtcttccttcattccacaaataattacaaacaacctactgtgtgccaggcactattcttagcactggaaatacactagtgaagaagcagatgaggaccctgtttattgtttctctccaagaaattctccaagaatattgtttcttggagagaaataataaataaacaagacaatttctgaaagcaataagtgcaatcaagataattaaaggatgctaaagtgtgacttgtggggattgggagagagatgcacagacaatattaaagaggaggcattcgagctttgttgtgaacaccggaagtaacatgccgagcgcctgggggatggaaactcctatagcaccccacaggctaacagcaagcaggacaagacaaaaagggcaggtgggacatggtagagatggaccctacccaggaaacagctccatcagcatcttagcctgccccactctagccacacatacccacgtgtgctcctgagttcagtgtgcccacctcactcccacaccctcacatagacttggcaagagtaaggagggaactccatagagacattttacctatctcaggggagcagccacaaagaagcaagtcttgtaaaaggtcttttgcaaaggagagtgaacccagcaatgagagatccttaacagctagtgcccattagggggctaaacctaaagcctgggtggtgatggctcaaacgctaatgagtcagtgaatccttaccgaccccctggcctttataatctgaggcaactttggctgcagcccgggaatgtgcagggcactagggaatacaaggccttcttccctggttgtcttgtaataaaacagccatggggttgtccctccagtccgagagactgtgatgaggcctacatagcagcgatgtggtcaggtaaaaatcaggaacccactgaaatcttgggcaagccaccctgcctgcttgtgcctcggttctctcatatgtcatatataggaggtgaggactccagctccacctgccccaggtgggtgtggtgatgatgaggaaagacaagaggcttgcaaggaccctgaagaggtcggagcatcatacagattcctttattagcccacattctgatgttccctggtgagacttgccccaagcaattgctagtaaatgggggttaatttcttctccacctccctactgaacaaaaaaagaaatgccagacttactaggagaatcgagttgctttgagtttcttttgttttgttttgttttgttttgttttaaggctccccttacacaccctcctttaagctttgggttttctctcttatagtttgttgacacatgctaaaaatgtctttggagagaacttctgcctgataaacacccaattctagactgtgggtggattttcgagctgacggtggtcaattcctttcattaagcagtgatctgatttctccacatggccattctgccttcttgggggcagagtagatgggcagcagttcaccttttcagagaaagaggtcttctagccacctgggctgctactgaatggttttctccaggacgctctacctaatgattatttctataacattaagcatggtaataagtagcttccaattcaattcatcctaaagccaaagaaaatacagcaacacacacacacacacacacacacacacacacacacacacacacacaccactttatggcaattcttaactgacattcaatgacttacttcttttcttagaaaatttccaccacatttctatccccaagccaacatacaatgtgaaatgaaagccagtgcgtggagtgcagctgctaaaaattttcagcacagggctctttctgactctgctcatgagatggtatcagccacccaatgactggcgtatcttggtcctgtgtctttcttcttacgctgtgttaatgtgtttactttccatttggcagagagacaagagagacacctccaacttcgacaaagagttcaccagacagcctgtggaactgacccccactgataaactcttcatcatgaacttggaccaaaatgaatttgctggcttctcttatactaacccagagtttgtcattaatgtgtaggtgaatgcaaactccatcgttgagcctggggtgtaagacttcaagccaagcgtatgtatcaattctagtcttccaggattcacggtgcacatgctggcattcaacatgtggaaagcttgtcttagagggcttttctttgtatgtgtagcttgctagtttgttttctacatttgaaaatgtttagtttagaataagcgcattatccaattatagaggtacaattttccaaacttccagaaactcatcaaatgaacagacaatgtcaaaactactgtgtctgataccaaaatgcttcagtatttgtaatttttcaagtcagaagctgatgttcctggtaaaagtttttacagttattctataatatcttctttgaatgctaagcatgagcgatatttttaaaaattgtgagtaagctttgcagttactgtgaactattgtctcttggaggaagttttttgtttaagaattgatatgattaaactgaattaatatatgcaa

SEQ ID NO: 16 - DNA, 1477 bp, Homo Sapiens
  mRNA (1)..(1477): gi|392306954|ref|NM_001267798.1| Homo sapiens protein tyrosine phosphatase, receptor type, C (PTPRC), transcript variant 5, mRNA
agaacaacttttttgacttcctgcaaagaggacccttacagtatttttggagaagttagtaaaaccgaatctgacatcatcacctagcagttcatgcagctagcaagtggtttgttcttagggtaacagaggaggaaattgttcctcgtctgataagacaacagtggagaaaggacgcatgctgtttcttagggacacggctgacttccagatatgaccatgtatttgtggcttaaactcttggcatttggctttgcctttctggacacagaagtatttgtgacagggcaaagcccaacaccttcccccactggccatctgcaagctgaggagcaaggaagccaatccaagtcaccaaacctcaaaagtagggaagctgacagttcagccttcagttggtggccaaaggcccgagagcccctcacaaaccactggagtaagtccaagagtccaaaagctgaggaacttggagtctgatgttcaagagcaggaagcagccagcacgagagaaagatgaagaccagaagactcagcaagctcacttctcctaccttcttgtgcctgctttttctagccgtgctggcagttgcttggatgatgcccactcatattgggtgggggtgggggggttggggagggtctgcctcccccagtccactgactcaaatgttaatctcccttggcaatacgctcacaggcacacccaggaacaatactttgcatccttcaatccaatcaagttgacactcaatattaaccatcaaatactattataaggagaatgttgcatgattttccttctagtctgtttgtaattcacatctaatgaaagagtgagagtggacgataaagggaacttgttgaaacatttctctcaaagcaaaagggatcattggaagcaggcagacaccagaattggtttaacctaaaaataacaaattaataattatcaagtctataatgatgacagtgacttaatgtgaatagaaagaattctaaactctctccttccttcctccctcccttctttcctactttctttccactccctttctcccacccccttttcttttcctttcttttctcccaccctctctccctccctttcttttattcaatgcatagtagttgaaaaaatctaaagttagacctgattttacactgaagactagaggtagttactatcctattactgtacttagttggctatgctggcatgtcattatgggtaaaagtttgatggatttatttgtgagttatttggttatgaaaatctagagattgaagtttttcattagaaaataacacacataacaagtctatgatcattttgcatttctgtaatcacagaatagttctgcaatatttcatgtatattggaattgaagttcaattgaattttatctgtatttagtaaaaattaactttagctttgatactaatgaataaagctgggttttttatttaaaaaaaaaaa

SEQ ID NO: 17 - DNA, 2514 bp, Homo Sapiens
  mRNA (1)..(2514): gi|295054104|ref|NM_001114937.2| Homo sapiens SH2 domain containing 1A (SH2D1A), transcript variant 2, mRNA
attactaagcatccccgtctgagtgcaagggtgtgtgttggcagtacagccccaatcttgcaaaatccttcttccaatgttcctcccctctctgtatgaaccctgtgttggggggcagaagatggaagcccttggcaagctcgatcgaaccaagctactaaattgctgagctcgttttaactgaagtgtgagaaggaggtttaaggcaagtagacaacatcctgttgttggggtgcttctctcttttttgcacatctggctgaactgggagtcaggtggttgacttgtgcctggctgcagtagcagcggcatctcccttgcacagttctcctcctcggcctgcccaagagtccaccaggccatggacgcagtggctgtgtatcatggcaaaatcagcagggaaaccggcgagaagctcctgcttgccactgggctggatggcagctatttgctgagggacagcgagagcgtgccaggcgtgtactgcctatgtgtgctgtatcacggttacatttatacataccgagtgtcccagacagaaacaggttcttggagtgctgagacagcacctggggtacataaaagatatttccggaaaataaaaaatctcatttcagcatttcagaagccagatcaaggcattgtaatacctctgcagtatccagttgagaagaagtcctcagctagaagtacacaagggataagagaagatcctgatgtctgcctgaaagccccatgaagaaaaataaaacaccttgtactttattttctataatttaaatatatgctaagtcttatatattgtagataatacagttcggtgagctacaaatgcatttctaaagccattgtagtcctgtaatggaagcatctagcatgtcgtcaaagctgaaatggacttttgtacatagtgaggagctttgaaacgaggattgggaaaaagtaattccgtaggttattttcagttattatatttacaaatgggaaacaaaaggataatgaatactttataaaggattaatgtcaattcttgccaaatataaataaaaataatcctcagtttttgtgaaaagctccatttttagtgaaatattattttatagctactaattttaaaatgtcttgcttgattgtatggtgggaagttggctggtgtcccttgtctttgccaagttctccactagctatggtgtcataggctcttttgggatttttgaagctgtatactgtgtgctaaaacaagcactaaacaaagagtgaaggatttatgtttaattctgaaagcaaccttcttgcctagtgttctgatattggacagtaaaatccacagaccaacctggagttgaaaatcttataatttaaaatatgctctaaacatgtttatcgtatttgatgctacaggatttgaaattgtattacaaatccaatgaaatgagtttttcttttcatttacctctgccccagttgtttctactacatggaagacctcattttgaagggaaatttcagcagctgcagctcatgagtaactgatttgtaacaagcctccttttaaagtaaccctacaaaaccactggaaagtttatggttgtattattttttaaaaaaattccaagtgattgaaacctacacgagatacagaattttatgcggcattttcttctcacatttatatttttgtgattttgtgattgattatatgtcactttgctacagggctcacagaattcattcactcaacaaacataatagggcgctgagggcatagaagtaaaaacacctggtccctgctctcagttcactgtcttgttggacgagaaaacaataacgataaaagacagtgaaagaaaataacgataaaagacagtgaaagaaaataacaataaaagacaaggaaaaaataacaatgaaagttgataagtacatgataagcgaggttccccgtgtgtaggtagatctggtctttagaggcagatagataggtcagtgcaaatactctggtccatgggccatatgaaaaggctaagcttcactgtaaaataataactgggaattctggattgtgtatgggtgttggtgaacttggttttaattagtgaactgctgagagacagagctattctccatgtactggcaagacctgatttctgagcatttaatatggatgccgtgggagtacaaaagtggagtgtggcctgagtaatgcattatgggtggtttaccatttcttgaggtaaaagcatcacatgaacttgtaaaggaatttaaaaatcctactttcataataagttgcataggtttaataatttttaattatatggcttgagtttaaattgtaataggcgtaactaattttaactctataatgtgttcattctggaataatcctaaacatatgaattatgtttgcatgttcacttccaagagcctttttttgaaaaaaagctttttttgaatcatcaagtctttcacatttaaataaagtgtttgaaagctttatttacctaaaaaaaa

SEQ ID NO: 18 - DNA, 25 bp, Homo Sapiens
  primer_bind (1)..(25): APOBEC3G>probeHG-U133A204205_at187301; Interrogation_Position=1177; Antisense;
gcccgcatctatgatgatcaaggaa

SEQ ID NO: 19 - DNA, 25 bp, Homo Sapiens
  primer_bind (1)..(25): CCL5>probeHG-U133A1405_i_at53877; Interrogation_Position=950; Antisense;
agcttccccaactaaagcctagaag

SEQ ID NO: 20 - DNA, 25 bp, Homo Sapiens
  primer_bind (1)..(25): CCR2>probeHG-U133A206978_at39605; Interrogation_Position=1661; Antisense;
tccatcgctgtcatctcagctggat

SEQ ID NO: 21 - DNA, 25 bp, Homo Sapiens
  primer_bind (1)..(25): CD2>probeHG-U133A205831_at981; Interrogation_Position=1006; Antisense;
agacctcgagttcagccaaaacctc

SEQ ID NO: 22 - DNA, 25 bp, Homo Sapiens
  primer_bind (1)..(25): CD27>probeHG-U133A206150_at246267; Interrogation_Position=661; Antisense;
cctgtgcagctccgattttattcgc

SEQ ID NO: 23 - DNA, 25 bp, Homo Sapiens
  primer_bind (1)..(25): CD3D>probeHG-U133A213539_at356491; Interrogation_Position=265; Antisense;
gggaacactgctctcagacattaca

SEQ ID NO: 24 - DNA, 25 bp, Homo Sapiens
  primer_bind (1)..(25): CD52>probeHG-U133A204661_at688107; Interrogation_Position=14; Antisense;
acagccacgaagatcctaccaaaat

SEQ ID NO: 25 - DNA, 25 bp, Homo Sapiens
  primer_bind (1)..(25): CORO1A>probeHG-U133A209083_at611665; Interrogation_Position=1020; Antisense;
tatctctccatgttcagttccaagg

SEQ ID NO: 26 - DNA, 25 bp, Homo Sapiens
  primer_bind (1)..(25): CXCL9>probeHG-U133A203915_at256427; Interrogation_Position=1973; Antisense;
gattatcaattaccacaccatctcc

SEQ ID NO: 27 - DNA, 25 bp, Homo Sapiens
  primer_bind (1)..(25): GZMA>probeHG-U133A205488_at577185; Interrogation_Position=373; Antisense;
cagccacacgcgaaggtgaccttaa

SEQ ID NO: 28 - DNA, 25 bp, Homo Sapiens
  primer_bind (1)..(25): GZMK>probeHG-U133A206666_at355123; Interrogation_Position=471; Antisense;
aaacctctcttagatctggaaccaa

SEQ ID NO: 29 - DNA, 25 bp, Homo Sapiens
  primer_bind (1)..(25): HLA-DMA>probeHG-U133A217478_s_at667235; Interrogation_Position=438; Antisense;
ctgttttgtcagtaatctcttccca

SEQ ID NO: 30 - DNA, 25 bp, Homo Sapiens
  primer_bind (1)..(25): IL2RG>probeHG-U133A204116_at469697; Interrogation_Position=867; Antisense;
ttctggctggaacggacgatgcccc

SEQ ID NO: 31 - DNA, 25 bp, Homo Sapiens
  primer_bind (1)..(25): LCK>probeHG-U133A204890_s_at48793; Interrogation_Position=1017; Antisense;
ctagtggattttctcaagacccctt

SEQ ID NO: 32 - DNA, 25 bp, Homo Sapiens
  primer_bind (1)..(25): PRKCB>probeHG-U133A207957_s_at330701; Interrogation_Position=2118; Antisense;
ttgctggcttctcttatactaaccc

SEQ ID NO: 33 - DNA, 25 bp, Homo Sapiens
  primer_bind (1)..(25): PTPRC>probeHG-U133A207238_s_at636103; Interrogation_Position=3732; Antisense;
acattcgagcaatatcaattcctat

SEQ ID NO: 34 - DNA, 25 bp, Homo Sapiens
  primer_bind (1)..(25): SH2D1A>probeHG-U133A210116_at423243; Interrogation_Position=1679; Antisense;
gaggttccccgtgtgtaggtagatc